(19) United States
(12) Patent Application Publication
(10) Pub. No.: US 2025/0305059 A1
(43) Pub. Date: Oct. 2, 2025

(54) PREDICTIVE BIOMARKERS IN COLORECTAL CANCER

(71) Applicant: UNIVERSITY OF SOUTH FLORIDA, Tampa, FL (US)

(72) Inventors: Timothy J. Yeatman, Tampa, FL (US); Mingli Yang, Tampa, FL (US)

(21) Appl. No.: 19/091,235

(22) Filed: Mar. 26, 2025

Related U.S. Application Data
(60) Provisional application No. 63/570,021, filed on Mar. 26, 2024.

(51) Int. Cl. C12Q 1/6886

(52) U.S. Cl. CPC C12Q 1/6886; C12Q 2600/112; C12Q 2600/158

(57) ABSTRACT

Disclosed herein are methods and compositions for quantifying distinct prognostic and predictive contributions of tumor epithelium vs. tumor microenvironment in colorectal cancer to optimize therapy.

### CROSS REFERENCE TO RELATED APPLICATIONS

This application claims the benefit of priority to U.S. Provisional Patent Application No. 63/570,021 filed on Mar. 26, 2024, the disclosure of which is expressly incorporated by reference herein in its entirety.

### STATEMENT REGARDING FEDERALLY SPONSORED RESEARCH

This invention was made with government support under grants R21CA256372, R21CA255312, U01CA157960, UH2CA227955, and UH3CA227955 awarded by the National Institutes of Health. The government has certain rights in the invention.

### FIELD

Disclosed herein are methods and compositions for quantifying distinct prognostic and predictive contributions of tumor epithelium vs. tumor microenvironment in colorectal cancer to optimize therapy.

### BACKGROUND

Colorectal cancer is the third most commonly diagnosed cancer in the United States, with around 150,000 cases diagnosed each year, and is also the third largest cause of cancer-related deaths. A quarter of patients treated for node-negative colorectal cancer by surgery alone are thought to be “cured” but will experience recurrence within five years. Currently, National Comprehensive Cancer Network (NCCN) Clinical Practice Guidelines are used to predict the risk of recurrence in colorectal cancer patients. Improved techniques for identifying patients at higher risk of cancer recurrence are needed to achieve better treatment plans and patient outcomes by better prediction of risk.

Current prognostic strategies for CRC primarily rely on tumor staging, histopathological analysis, and molecular biomarkers, such as KRAS, NRAS, and BRAF mutations, as well as microsatellite instability (MSI) status. These biomarkers help guide therapeutic decisions, particularly in the selection of targeted therapies and immunotherapies. Standard therapeutic approaches for CRC include surgical resection, chemotherapy, targeted therapy, and immunotherapy. First-line treatment for metastatic CRC typically involves combination chemotherapy regimens, such as FOLFOX (fluorouracil, leucovorin, and oxaliplatin) or FOLFIRI (fluorouracil, leucovorin, and irinotecan), with or without biologic agents targeting epidermal growth factor receptor (EGFR) or vascular endothelial growth factor (VEGF). More recently, immune checkpoint inhibitors, such as pembrolizumab and nivolumab, have been introduced for MSI-high or mismatch repair-deficient (dMMR) CRC patients. While these therapies have improved patient outcomes, they do not fully account for the influence of the tumor microenvironment (TME), which plays a critical role in shaping drug sensitivity and resistance. Consequently, a more comprehensive approach that integrates both tumor cells and tumor microenvironment contributions is needed to enhance prognostic accuracy and optimize therapeutic strategies for CRC patients. Despite efforts to develop predictive biomarkers and targeted therapies, a more comprehensive approach is needed to improve patient stratification and treatment outcomes.

Furthermore, the cellular interactions within the tumor microenvironment and their role in modulating response to targeted therapies have not been fully elucidated. Therefore, there is a need for innovative methods and analytical frameworks that accurately distinguish and quantify the contributions of tumor epithelium and the tumor microenvironment in cancer.

### SUMMARY

Disclosed herein are methods and biomarker panels for treating colorectal cancer (CRC), determining CRC therapy response, and guiding treatment selection in a subject based on gene expression signatures.

In some examples, disclosed herein is a method of determining, calculating, computing, identifying, detecting, measuring, evaluating, assessing, deriving, and/or ascertaining colorectal cancer (CRC) related therapy response, comprising obtaining a biological sample from a CRC subject, measuring expression levels of a 10-gene tumor microenvironment signature (TME^{S}), a 10-gene epithelial signature (EPI^{S}), or a combination thereof in the biological sample, calculating a TME^{S} score, EPI^{S} score or combination thereof, classifying the CRC subject based on the TME^{S} score or the EPI^{S} score, wherein a high TME^{S} score indicates worse survival and resistance to CRC targeted therapy immunotherapy and MEK inhibitors, wherein a high EPI^{S} score indicates better survival and sensitivity to CRC targeted therapies, and administering a therapeutically effective dose of CRC targeted therapy to the CRC subject.

In some examples, the CRC targeted therapy comprises immunotherapy, EGFR inhibitors, SRC inhibitors or MEK inhibitors.

In some examples, the TME^{S} score and the EPI^{S} score are calculated by quantifying gene expression levels of 10-gene TME^{S} and 10-gene EPI^{S} signatures, respectively.

In some examples, the 10-gene epithelial signature of any preceding aspect is expressed in epithelial tumor cells or epithelial normal mucosa. In some examples, the EPI^{S} score is calculated using gene expression levels of 10-gene epithelial signature.

In some examples, the 10-gene tumor microenvironment signature is expressed in stromal or immune cells in tumor microenvironment. Also disclosed herein, in some examples, the TME^{S} score is calculated using gene expression levels of 10-gene tumor microenvironment signature.

In some examples, the biological sample comprises a surgical resection specimen, tissue biopsy (such as, for example, tissue section, fixed sample, formalin fixed, paraffin embedded (FFPE) sample) or fine needle aspirate.

In some examples, the immunotherapy agents comprise such as, for example, including but not limited to pembrolizumab (KEYTRUDA™), nivolumab (OPDIVO®), ipilimumab (YERVOY®), atezolizumab (TECENTRIQ®), or dostarlimab (JEMPERLI™), primarily targeting immune checkpoints like PD-1, PD-L1, or CTLA-4, offering effective treatment for microsatellite instability-high (MSI-H) or mismatch repair-deficient (dMMR) CRC cases. In some examples, the cancer-associated fibroblast (CAF)-related therapy comprises such as, for example, including but not limited fibroblast activation protein (FAP) inhibitors (such as, for example, talabostat), TGFβ inhibitors (such as, for example, galunisertib), CXCL12/CXCR4 inhibitors (such as, for example, plerixafor). Disclosed herein, in some examples administering a therapeutically effective amount of immunotherapy (checkpoint inhibitor therapy), SRC inhibitor therapy or cancer-associated fibroblast (CAF)-related therapy to a subject with high TME^{S} score.

In some examples, the EGFR inhibitors comprise such as, for example, including but not limited to cetuximab (ERBITUX®) or panitumumab (VECTIBIX®), blocking EGFR signaling and are used in patients with RAS wild-type tumors. In some examples, the MEK inhibitors comprise such as, for example, including but not limited to trametinib (MEKINIST®), binimetinib (MEKTOVI®), or selumetinib (KOSELUGO®), acting on the MAPK/ERK signaling pathway, offering benefits in CRC cases with mutations in the RAS-RAF pathway. These agents are used either as monotherapies or in combination with other treatments to enhance efficacy and improve patient outcomes. Disclosed herein, in some examples administering a therapeutically effective amount of MEK inhibitors or EGFR inhibitors to a subject with high EPI^{S} score.

In some examples, the 10-gene tumor microenvironment signature comprises CD109, AHNAK2, GAS1, PRKCDBP (CAVIN-3), MEIS2, NXN, GFPT2, PMP22, WWTR1 or PTRF (CAVIN-1) gene.

In some examples, the 10-gene epithelial signature comprises CDX1, CDX2, C10orf99, DDC, GPA33, FAM84A (LRATD1), NR1I2, MYB, C2orf89 (TRABD2A) or EPHB2 gene.

In some examples, the CRC subject is classified into at least one of consensus molecular subtypes (CMS) comprising of a CMS1 subtype, a CMS2 subtype, a CMS3 subtype, and a CMS4 subtype. Disclosed herein, the CRC subtypes are based on distinct molecular and pathological characteristics.

In some examples, the CMS1 subtype or the CMS4 subtype is correlated to worse survival in the CRC subject. In some examples, the CMS1 subtype and the CMS4 subtype is correlated with the TME^{S} score. Also disclosed herein, the CMS1 subtype of any preceding aspect is positively correlated with memory B cells, CD8+ T cells, gamma delta T cells, NK cells, macrophages or dendritic cells. Disclosed herein, the CMS4 subtype of any preceding aspect is positively correlated with stromal cells or tumor cells.

In some examples, the CMS2 subtype or the CMS3 subtype is correlated to better survival in the CRC subject. In some examples, the CMS2 subtype and the CMS3 subtype are correlated with the EPI^{S} score. Also disclosed herein, the CMS2 subtype and the CMS3 subtype of any preceding aspect are correlated with inactive B cells, resting NK cells and macrophages (M0).

In some examples, disclosed herein is a method for treating, inhibiting, reducing, decreasing, ameliorating, and/or preventing CRC in a subject, comprising obtaining a biological sample from the subject, measuring expression levels of a 10-gene tumor microenvironment signature (TME^{S}), wherein the TME^{S} comprises CD109, AHNAK2, GAS1, PRKCDBP, MEIS2, NXN, GFPT2, PMP22, WWTR1, or PTRF gene, calculating a TME^{S} score, analyzing presence of CRC subtype CMS4 or subtype CMS1 based on a high TME^{S} score, and administering a therapeutically effective amount of immunotherapy (checkpoint inhibitor therapy), SRC inhibitor therapy or cancer-associated fibroblast (CAF)-related therapy to the subject.

In some examples, disclosed herein is a method for treating, inhibiting, reducing, decreasing, ameliorating, and/or preventing CRC in a subject, comprising obtaining a biological sample from the subject, measuring expression levels of 10-gene epithelial-associated signature (EPI^{S}), wherein the EPI^{S} comprises CDX1, CDX2, C10orf99, DDC, GPA33, FAM84A, NR1I2, MYB, C2orf89, or EPHB2 gene, calculating an EPI^{S} score, analyzing presence of CRC subtype CMS2 or subtype CMS3 based on a high EPI^{S} score, and administering a therapeutically effective amount of EGFR inhibitor (EGFRi) therapy to the subject.

In some examples, disclosed herein is a method of stratifying, classifying, segmenting, categorizing, grouping, dividing, differentiating, and/or sorting a subject with CRC for targeted therapy selection, comprising obtaining a biological sample from the subject, performing single-cell RNA sequencing (scRNA-seq) analysis on the biological sample, identifying TME^{S}-positive or EPI^{S}-positive cells based on gene expression signatures, calculating a TME^{S} score or an EPI^{S} score, administering a therapeutically effective amount of immunotherapy, SRC inhibitor therapy or cancer-associated fibroblast (CAF)-related therapy to the subject with high TME^{S} score, and administering a therapeutically effective amount of EGFRi therapy or MEK inhibitor therapy to the subject with high EPI^{S} score.

In some examples, disclosed herein is a combined biomarker panel for determining treatment option in a CRC subject, comprising a 10-gene epithelial-associated signature (EPI^{S}), wherein the EPI^{S} comprises CDX1, CDX2, C10orf99, DDC, GPA33, FAM84A (LRATD1), NR1I2, MYB, C2orf89 (TRABD2A) or EPHB2 genes, and a 10-gene tumor microenvironment signature (TME^{S}), wherein the TME^{S} comprises CD109, AHNAK2, GAS1, PRKCDBP (CAVIN3), MEIS2, NXN, GFPT2, PMP22, WWTR1 or PTRF (CAVIN1)genes, wherein gene expression levels of EPI^{S} or TME^{S} are measured in a sample obtained from the CRC subject.

### BRIEF DESCRIPTION OF THE DRAWINGS

The accompanying figures, which are incorporated in and constitute a part of this specification, illustrate several examples described below.

FIGS. 1A, 1B, 1C, 1D, 1E, and 1F show the highly prognostic ΔPC1. EMT score was strongly associated with both CMS1 and CMS4 subtypes independent of stages, primary/metastatic tumor types, and MSI/MSS status in the Merck-Moffitt CRCs. FIGS. 1A-1C show the ΔPC1.EMT score comparison and Kaplan-Meier (KM) survival analyses by the score quartiles (Q1-Q4) in Merck-Moffitt CRCs that had corresponding overall survival (OS) data. FIGS. 1D-1F. The ΔPC1.EMT score comparison among the CMS1 vs. CMS2 vs. CMS 3 vs. CMS4 subtypes. Here the CMS1-4 subtypes were generated by CMScaller. FIGS. 1A and 1D. all stages and individual stages (Stage I* (including 6 Stage 0 cases), Stage II, Stage III and Stage IV); FIGS. 1B and 1E. primary and metastatic tumors; FIGS. 1C and 1F. MSI and MSS tumors. Bars represent Median with interquartile range. Adjusted P values are shown for two-tailed Welch's t test after adjustments for multi-comparisons by Holm-Bonferroni method. See Table 1 for detailed description of the baseline phenotypic characteristics.

FIGS. 2A, 2B, 2C, 2D, 2E, 2F, 2G, and 2H show the ΔPC1.EMT's top 10 positively correlated (POS) genes (vs. top 10 negatively correlated (NEG) genes) were strongly correlated with the CMS1 and CMS4 (vs. the CMS2 and CMS3) subtypes that portended worse survival. Comparison of gene expression of (FIG. 2A) the ΔPC1.EMT's top 10 positively correlated (POS) genes and (FIG. 2B) top 10 negatively correlated (NEG) genes across the CMS1-4 subtypes. FIG. C shows comparison of the 10-gene POS and 10-gene NEG signature scores across the CMS1-4 subtypes. Here the CMS1-4 subtypes were generated by CMScaller. Bars represent Median with interquartile range. Adjusted P values are shown for two-tailed Welch's t test after adjustments for multi-comparisons by Holm-Bonferroni method. Spearman correlation of the 10 POS genes/score and 10 NEG genes/score with the CMS1*, CMS2*, CMS3* and CMS4* scores in (FIG. 2E) the TCGA (n=626) and (FIG. 2G) the Marisa (n=566) CRC datasets, respectively. The CMS1*, CMS2*, CMS3* and CMS4* scores were designated to measure the propensity of a tumor to fall into CMS1, CMS2, CMS2 and CMS4 classes, respectively. The Kaplan-Meier (KM) survival analyses among CMS1, CMS2, CMS3 and CMS4 subtypes in (FIG. 2D) Merck-Moffitt (n=2009, OS), (FIG. 2F) TCGA (n=577, OS) and (FIG. 2H) Marisa (n=489, RFS) tumors, respectively.

FIG. 3 shows CIBERSORT deconvolution and spatial transcriptomics revealed that the 10 POS genes/score vs. the 10 NEG genes/score were distinctly associated with the epithelial tumor vs. immune/stromal TME cellular features. Spearman correlation of the 10 POS genes/score and 10 NEG genes/score with the 20 CIBERSORT scores and the CMS1*, CMS2*, CMS3* and CMS4* scores in the Merck-Moffitt CRC tumors (n=2373). The 20 CIBERSORT cell scores were derived from the deconvolution analysis of Affymetrix gene expression data to measure the abundances of various immune cell populations as well as those of “stromal” cells and “tumor” cells, respectively.

FIGS. 4A, 4B, 4C, 4D, and 4E show the single cell expression analysis using an independent public scRNASEQ dataset validating that the 10 POS genes were principally TME-associated genes, and the 10 NEG genes were predominantly EPI-associated genes. The scRNASEQ dataset (n=62) was reported to transcriptionally profiled 371,223 cells (258,359/112,864 cells, T/N) from colorectal tumors and adjacent normal tissues of 62 CRC patients. The 10 POS genes and the 10 NEG genes were analyzed using the data of all cells on the Human Colon_Cancer Atlas (c295)—Single Cell Portal (broadinstitute.org). The t-distributed stochastic neighbor embedding (t-SNE) of cell subsets are illustrated for (FIG. 4A) clustered by 7 major cell classes, (FIG. 4B) clustered by 20 cell subclasses and (FIG. 4C) clustered by tumor vs. normal cells. The single cell expression is shown for each of (FIG. 4D) the 10 POS genes and (FIG. 4E) the 10 NEG genes and both t-SNE and dot plots are shown. The POS gene expression in the tumor fibroblasts are highlighted by red circles in FIGS. 4B and 4D.

FIGS. 5A, 5B, 5C, 5D, 5E, 5F, and 5G show the TME^{S} vs. the EPI^{S} genes and their respective signature scores portended distinct prognostic outcomes (worse vs. better OS). The Kaplan-Meier (KM) survival analyses by the gene expression quartiles (Q1-Q4) of (FIG. 5A) the 10 TME^{S} genes and (FIG. 5B) the 10 EPI^{S} genes in the Merck-Moffitt CRC tumors that had corresponding OS data (n=2246). Notably, 9 of the 10 TME^{S} genes portended worse OS, whereas all the 10 EPI^{S} genes portended better OS. The KM analyses by the score quartiles (Q1-Q4) of the 10-gene TME^{S} score, the 10-gene EPI^{S} score and their 20-gene TME^{S}-EPI^{S} composite score in (FIG. 5C) all stage tumors (n=2246), (FIG. 5D) Stage I-III primary tumors (n=1289), (FIG. 5E) Stage IV tumors (n=730), (FIG. 5F) MSI tumors (n=230), (FIG. 5G) MSS tumors (n=2016), respectively.

FIGS. 6A, 6B, and 6C show the Cox proportional hazards regression analyses of the 10-gene TME^{S} score and the 10-gene EPI^{S} score, along with 29 TME-functional gene expression signatures (Fges), in the Merck-Moffitt CRC tumors that had corresponding OS data (n=2246). FIG. 6A shows univariable Cox regression analysis. FIG. 6B shows multivariable Cox regression analysis. FIG. 6C shows bivariable Cox regression analyses of the 10-gene TME^{S} score against each of the 29 TME-Fges, respectively (total 29 pairs). OR odd ratio; HR, hazard ratio. Here X10_gene_TME_S_score represents the 10-gene TME^{S} score, and X10_gene_EPI_S_score represents the 10-gene EPI^{S} score.

FIGS. 7A, 7B, 7C, and 7D show the 10-gene TME^{S} signature score (vs. the 10-gene EPI^{S} signature score) strongly correlated with the EMT, SRC activation and MEK inhibitor (MEKi) resistance signature scores in multiple CRC datasets. Scatter plots of (FIG. 7A) the 10-gene TME^{S} score and (FIG. 7B) the 10-gene EPI^{S} score versus the EMT, SRC activation, 13-gene MEKi resistance and 18-gene MEK pathway activation signature scores, respectively, in the Merck-Moffitt CRC tumors (n=2373). Spearman r and P values are shown. The CMS1-4 subtypes are indicated by red (CMS4) vs. orange (CMS3) vs. green (CMS2) vs. blue (CMS1) colors. (FIGS. 7C and 7D) Similar scatter plots were made in an independent (Marisa) CRC dataset (n=566). Here the CMS1-4 subtypes were generated by CMScaller. Scatter plots were also made in the Merck-Moffitt Stage IV tumors (n=808) (FIGS. 17A and 17B). Spearman correlation heatmaps of the individual TME^{S} and EPI^{S} genes with the EMT, SRC activation, 13-gene MEIk resistance and 18-gene MEK pathway activation signature scores in the Merck-Moffitt (n=2373), TCGA (n=626) and Marisa (n=566) CRC datasets are shown in FIGS. 18, 19 and 20, respectively.

FIGS. 8A, 8B, 8C, and 8D show the retrospective analysis of two independent clinical trial datasets showed that the 10-gene EPI^{S} signature score significantly predicted longer progression free survival (PFS) in cetuximab (CTX)-treated metastatic CRC patients. The Kaplan-Meier (KM) survival analyses of the 10-gene EPI^{S}, the 10-gene TME^{S}, and 20-gene EPI^{S}-TME^{S} signature scores in (FIG. 8A) the Khambata-Ford CTX-treated metastatic CRC patients (n=80, all cases regardless of KRAS mutation status) and (FIG. 8B) a subset of WT KRAS patients (n=43) as well as (FIG. 8D) Merck-PN04 CTX-treated WT KRAS metastatic CRC patients. FIG. 8C shows comparison of the 10-gene EPI^{S}, the 10-gene TME^{S}, or 20-gene TME^{S}-EPI^{S} scores among Khambata-Ford CR/PR (complete response/partial response), SD (stable disease), PD (progressed disease) and UTD (undetermined) patients. Bars represent Median with interquartile range. Unadjusted P values are for two-tailed Welch's t test; ns-not significant. Of note, those significant unadjusted p values remained significant after adjustments for multi-comparisons by Holm-Bonferroni method. Note: *3 EPI^{S} genes (C10orf99, FAM84A, TRABD2A) and 1 TME^{S} gene (CD109) did not have probe values in the Khambata-Ford dataset, so these 4 genes were excluded in the analyses.

FIGS. 9A, 9B, 9C, 9D, 9E, 9F, 9G, and 9H show the 10-gene EPI^{S}(vs. the 10-gene TME^{S}) signature scores were independently validated as mildly prognostic, but highly predictive of EGFRi outcomes using a large real world CarisLS CRC dataset. The Kaplan-Meier (KM) survival analyses of (FIGS. 9A-9D) the 10-gene EPI^{S} and (FIGS. 9E-9H) the 10-gene TME^{S} score quartiles (Q4-purple, highest; Q3—green; Q2—red; Q1—blue, lowest) were performed in the CarisLS (FIGS. 9A and 9E) all non-EGFRi CRC tumors (n=11369, OS), and (FIGS. 9B and 9F) EGFRi-treated (n=2343, OS), (FIGS. 9C and 9G) cetuximab (CTX)-treated (n=953, TOT) and (FIGS. 9D and 9H) panitumumab (PMB)-treated (n=1307, TOT) tumors, respectively. EGFR inhibitor (EGFRi) therapies include both CTX and PMB. OS-overall survival (OS); TOT-time on treatment.

FIG. 10 shows a flowchart of the study illustrating generation and analysis of the 10-gene TME^{S} and the 10-gene EPI^{S} signature scores using the Merck-Moffitt CRC dataset and various other CRC datasets. The studies using the Merck-Moffitt dataset are highlighted by light yellow color, whereas the studies using other independent datasets are highlighted by light blue color.

FIGS. 11A, 11B, 11C, 11D, 11E, 11F, 11G, and 11H show the ΔPC1.EMT score was strongly associated with both CMS1 and CMS4 subtypes independent of age, race, sex and sidedness in the Merck-Moffitt CRCs. The ΔPC1.EMT score comparison among the subgroups of age (FIG. 11A), race (FIG. 11C), sex (FIG. 11E) and sidedness (FIG. 11G). The ΔPC1.EMT score comparison among the CMS1 vs. CMS2 vs. CMS 3 vs. CMS4 subtypes in the patients of (FIG. 11B)<50 yr and >50+ yr, (FIG. 11D) African American/Black and White, (FIG. 11F) female and male, and (FIG. 11H) left- and right-sided, respectively. Here the CMS1-4 subtypes were generated by CMScaller. Bars represent Median with interquartile range. Adjusted P values are shown for two-tailed Welch's t test after adjustments for multi-comparisons by Holm-Bonferroni method. See Table 1 for detailed description of the baseline phenotypic characteristics.

FIGS. 12A, 12B, 12C, 12D, and 12E show the Spearman correlation of the CMS subtypes generated by the CMScaller, the random forest (rf) and the single-cell predictor (ssp) in the Merck-Moffitt CRC tumors (n=2373). Comparison of the 10-gene POS and 10-gene NEG signature scores across the CMS1-4 subtypes generated by the rf (FIG. 12B) and by the ssp (FIG. 12C). Bars represent Median with interquartile range. Adjusted P values are shown for two-tailed Welch's t test after adjustments for multi-comparisons by Holm-Bonferroni method. The Kaplan-Meier (KM) survival analyses among CMS1-4 subtypes generated by the rf (FIG. 12D) and by the ssp (FIG. 12E).

FIGS. 13A, 13B, and 13C show the TME^{S} genes/score vs. the EPI^{S} genes/score were distinctly correlated with BRAF (V600E) and APC truncating mutations. (FIG. 13A) Spearman correlation of the 10 TME^{S} and the 10 EPI^{S} genes, and their respective 10-gene signature scores with APC(truncating), KRAS, TP53, BRAF (V600E) and PIK3CA mutations, and with MSI-H as well as with the CMS1*, CMS2*, CMS3* and CMS4* scores in the Moffitt 468 CRC tumors. Only the BRAF(V600E) and APC truncating mutations were considered as functional mutations for these two driver genes. The 10-gene TME^{S} score and most of the TME^{S} genes were significantly correlated with BRAF (V600E) and MSI and anti-correlated with APC truncating mutations. By contrast, the 10-gene EPI^{S} score and most of the EPI^{S} genes were significantly correlated with APC truncating mutations and anti-correlated with BRAF (V600E) and MSI. (FIG. 13B) Comparison of the 10-gene TME^{S} score, and the 10-gene EPI^{S} score between BRAF (V600E) and BRAF WT (i.e. without BRAF (V600E)) or between KRAS-mutations and KRAS WT or between APC truncating mutations and APC WT (i.e. without APC truncating mutations) in the Moffitt 468 tumors. (FIG. 13C) Comparison of the TME^{S} and the EPI^{S} scores between BRAF (V600E) and BRAF WT or between KRAS-mutations and KRAS WT in an independent (Marisa) CRC dataset that had BRAF and KRAS mutation data. Bars represent Median with interquartile range. P values are for two-tailed Welch's t test.

FIGS. 14A and 14B show the Kaplan-Meier (KM) survival analyses by the score quartiles (Q1-Q4) of (FIG. 14A) the 10-gene TME^{S} signature score and (FIG. 14B) the 10-gene EPI^{S} signature score in the Marisa CRC tumors that had corresponding RFS data (n=557).

FIGS. 15A and 15B show Heatmap and Spearman Correlations. FIG. 15A shows the Heatmap correlation analysis on the iCMS2/iCMS3 signatures vs. the CMS subtypes in the Merck-Moffitt tumors (n=2373). FIG. 15B shows the Spearman correlation of the 10-gene TME^{S} score, and the 10-gene EPI^{S} score with the iCMS2 and iCMS3's up and down gene signature scores as well as their “up—down” signature scores in the Merck-Moffitt tumors (n=2373).

FIGS. 16A and 16B show the comparison of the 10-gene TME^{S} and 10-gene EPI^{S} signature scores across the PDS1-3 subtypes in the Merck-Moffitt CRC tumors. Adjusted P values are shown for two-tailed Welch's t test after adjustments for multi-comparisons by Holm-Bonferroni method. FIG. 16B shows the Kaplan-Meier (KM) survival analyses among PDS1-3 subtypes.

FIGS. 17A and 17B show the 10-gene TME^{S} signature score (vs. the 10-gene EPI^{S} signature score) strongly correlated with the EMT, SRC activation and MEK inhibitor resistance signature scores in the Merck-Moffitt Stage IV CRC tumors (n=808). Scatter plots of (FIG. 17A) the 10-gene TME^{S} score and (FIG. 17B) the 10-gene EPI^{S} score versus the EMT, SRC activation, 13-gene MEKi resistance and 18-gene MEK pathway activation signature scores, respectively. Spearman r and P values are shown. The CMS1-4 subtypes are indicated by colors (CMS4 vs. CMS3 vs. CMS2 vs. CMS1 vs. CMS-NA). Here the CMS1-4, NA subtypes were generated by CMScaller.

FIG. 18 shows the 10 TME^{S} genes (vs. the 10 EPI^{S} genes) strongly correlated with the EMT, SRC activation, 13-gene MEK inhibitor resistance signature scores in the Merck-Moffitt CRC tumors (n=2373). Spearman correlation heatmap among the expression of individual genes and the EMT, SRC activation, 13-gene MEKi resistance and 18-gene MEK pathway activation signature scores is shown.

FIG. 19 shows the 10 TME^{S} genes (vs. the 10 EPI^{S} genes) strongly correlated with the EMT, SRC activation, 13-gene MEK inhibitor resistance signature scores in an independent validation (TCGA) CRC dataset (n=626). Spearman correlation heatmap among the expression of individual genes and the EMT, SRC activation, 13-gene MEKi resistance and 18-gene MEK pathway activation signature scores is shown.

FIG. 20 shows the 10 TME^{S} genes (vs. the 10 EPI^{S} genes) strongly correlated with the EMT, SRC activation, 13-gene MEK inhibitor resistance signature scores in another independent validation (Marisa) CRC dataset (n=566). Spearman correlation heatmap among the expression of individual genes and the EMT, SRC activation, 13-gene MEKi resistance and 18-gene MEK pathway activation signature scores is shown.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

It has been suggested that tumorigenesis and therapeutic response may depend not only on tumor epithelium (EPI), but also on the tumor microenvironment (TME), composed of a variety of non-cancerous immune and stromal cells. Cancer progression and metastasis are thought to result from complex interactions between tumor cells and the TME. Colorectal cancer (CRC) is a highly heterogeneous disease that has diverse genetic, molecular and clinical features associated with metastasis, prognosis and therapeutic outcomes. While both the biology of the CRC tumor and its associated TME are both likely contributory to therapeutic clinical outcomes, the cellular contribution of the tumor epithelial cell versus the resident TME towards drug sensitivity and resistance has neither been clearly defined nor quantified.

A tumor comprises a heterogeneous mixture of epithelial tumor cells and immune and stromal cells within the TME. Historically, cancer therapies have predominantly targeted epithelial tumor cells, often neglecting the immune and stromal components of the TME. Conventional therapies such as chemotherapy, radiotherapy, and certain targeted therapies primarily aim at epithelial tumor cells, yet inadvertently also impact cells within the TME. Therapies designed exclusively against epithelial cells, such as epidermal growth factor receptor inhibitors (EGFRi), demonstrate effectiveness through targeted action solely on tumor cells.

Recent clinical evidence, particularly from rectal cancer treatment, highlights the therapeutic potential of targeting the TME itself. Rectal cancers previously treated primarily with chemotherapy, radiotherapy, and surgical interventions have shown curative responses with checkpoint inhibitors, monoclonal antibodies targeting T-cell receptors, in specific tumor subtypes. This underscores the emerging therapeutic importance of directly engaging the TME.

The disclosed tumor signatures enable assessment and differentiation of therapeutic potential and status between the epithelial tumor cells and the immune/stromal cells of the TME. By clearly distinguishing these two components, treatment can be effectively tailored to simultaneously target epithelial tumor cells and beneficially engage the TME. In contrast, conventional methods such as pre-operative radiotherapy (XRT) eliminate both tumor cells and local immune populations indiscriminately. This non-selective approach may contribute to aggressive and resistant tumor recurrences when residual disease persists.

Accordingly, the disclosed signatures can inform more precise and tailored therapeutic strategies that concurrently target epithelial tumor cells and modulate the immune/stromal TME. While checkpoint inhibitors currently represent an emerging approach, therapies such as bispecific antibodies, capable of targeting additional TME-associated molecules such as vascular endothelial growth factor (VEGF), underscores the increasing significance of therapeutically engaging the TME. The disclosed signatures thus provide crucial guidance for advanced therapeutic strategies aimed at optimizing both epithelial and TME-targeted treatment modalities.

Heterogenous CRC has been classified into four distinct consensus molecular subtypes (CMS): CMS1 subtype is characterized by microsatellite instability (MSI) or immune activation and is associated with worse survival after relapse (SAR); CMS2 subtype, of epithelial cell origin, is distinguished by WNT/MYC signaling pathways; CMS3 subtype, also of epithelial origin, is marked by dysregulated metabolism; CMS4 subtype, classified as mesenchymal, exhibits stromal infiltration, angiogenesis, and TGFP activation, correlating with worse overall survival (OS) and relapse-free survival (RFS). This classification system provides critical insights into CRC prognosis and treatment strategies. The CMS1-4 subtypes have been applied to immune-classify CRC: CMS1, immune activated; CMS2, immune desert; CMS3, immune excluded; CMS4, immune inflamed. In this study, 2373 human CRC tumors were classified into the CMS1-4 subtypes with distinct, variable TME cellular features defined by CIBERSORT deconvolution analysis of bulk gene expression data. scRNASEQ derived from an independent dataset documented the precise cellular origin of signature transcripts. The evidence is presented in this application, clearly demonstrating and quantifying the distinct cellular contributions of the EPI vs. the TME in determining CRC prognosis and therapeutic outcomes. Moreover, these analyses have resulted in the generation of a pair of new, distinct, predictive 10-gene signature scores (the TME^{S} score vs. the EPI^{S} score)biomarkers capable of quantifying the dependency of clinical outcomes on tumor epithelial cells vs. the TME, which may ultimately help optimize therapeutic strategies for CRC patients.

In one example, 2373 colorectal cancer (CRC) tumors were classified into the consensus molecular subtypes (CMS1-4) and generated the 10-gene TME^{S} and the 10-gene EPI^{S} signatures as the serendipitous derivatives of the most (positively vs. negatively) correlated genes of a highly-prognostic, ˜500-gene signature which was previously identified. Distinct TME vs. EPI cellular features of the signature genes were identified by CIBERSORT deconvolution and validated by scRNASEQ in an independent public dataset.

It was observed that the TME^{S} signature was strongly associated with the immune/stromal TME-rich CMS1/CMS4 subtypes that portended worse survival, whereas the EPI^{S} signature was predominantly related to the TME-poor, epithelial CMS2/CMS3 classes that portended better survival. Multivariable Cox regression analysis against 29 TME-related signatures revealed that the TME^{S} signature was the most strikingly impacted by the “Cancer-associated fibroblasts” signature (HR: 10.87 vs. 0.13, both P<0.0001). Moreover, the TME^{S} score was strongly correlated with EMT, SRC activation and MEK inhibitor resistance in 2373 CRC tumors (Spearman r=0.727, 0.802, 0.824, respectively), which was validated in two independent CRC datasets (n=626 and n=566). By contrast, the EPI^{S} score was the dominant force in associating with longer progression free survival in cetuximab-treated metastatic CRC patients derived from two independent clinical trials (Logrank trend P=0.0005/n=80; P=0.0013/n=44). This finding was further validated in a large real-world clinical-genomics dataset with EGFR inhibitor therapy, which demonstrated that higher EPI^{S} scores were associated with increased overall survival (EGFRi, Logrank trend P<0.0001/n=2343) and time on treatment (cetuximab, P=0.003/n=953; panitumumab, P<0.0001/n=1307).

It was identified that a pair of new, distinct 10-gene signatures (the EPI^{S} vs. the TME^{S}) is capable of distinguishing the cellular contribution of the tumor EPI vs. the TME in determining CRC prognosis and therapeutic outcomes. With targeted approaches emerging to address both tumor epithelial cells and the TME, the EPI^{S} vs. TME^{S} signature scores have a novel biomarker role to permit optimization of CRC therapy by identifying sensitive vs. resistant subpopulations.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

“Administration” to a subject or “administering” includes any route of introducing or delivering to a subject an agent. Administration can be carried out by any suitable route, including oral, intravenous, intraperitoneal, intranasal, inhalation and the like.

Administration includes self-administration and the administration by another.

The terms “about” and “approximately” are defined as being “close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

The term “cancer” or “neoplasms” used herein is meant to include all types of cancerous growths or oncogenic processes, metastatic tissues or malignantly transformed cells, tissues, or organs, irrespective of histopathologic type or stage of invasiveness. The terms “cancer” or “neoplasms” include malignancies of the various organ systems, such as malignancies affecting skin, brain, spinal cord, cervix, bladder, lung, breast, thyroid, lymphoid tissues, connecting tissues, gastrointestinal, and genito-urinary tracts, that include, but are not limited to, glioma, melanoma, lung cancer, breast cancer, cervical squamous cell carcinoma, bladder cancer, and soft tissue sarcoma. The term “cancer metastasis” has its general meaning in the art and refers to the spread of a tumor from one organ or part to another non-adjacent organ or part.

The term “comprising” and variations thereof as used herein, is used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples and are also disclosed.

A “composition” is intended to include a combination of active agent and another compound or composition, inert (for example, a detectable agent or label) or active, such as an adjuvant.

As used herein, the terms “determining,” “measuring,” and “assessing,” and “assaying” are used interchangeably and include both quantitative and qualitative determinations.

As used herein the term “encoding” refers to the inherent property of specific sequences of nucleotides in a nucleic acid, to serve as templates for synthesis of other molecules having a defined sequence of nucleotides (i.e. rRNA, tRNA, other RNA molecules) or amino acids and the biological properties resulting therefrom.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional properties, such as removing or adding amino acids capable of disulfide bonding to increase their bio-longevity, altering their secretory characteristics, etc. In any case, the functional fragment must possess a bioactive property, such as antigen binding and antigen recognition.

The term “gene” or “gene sequence” refers to the coding sequence or control sequence or fragments thereof. A gene may include any combination of coding sequence and control sequence or fragments thereof. Thus, a “gene” as referred to herein, may be all or part of a native gene. A polynucleotide sequence, as referred to herein, may be used interchangeably with the term “gene” or may include any coding sequence, non-coding sequence, or control sequence, fragments thereof, and combinations thereof. The term “gene” or “gene sequence” includes, for example, control sequences upstream of the coding sequence (for example, the ribosome binding site).

The term “isolating” as used herein refers to isolation from a biological sample, i.e., blood, plasma, tissues, exosomes, or cells. As used herein the term “isolated,” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to purification.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22: 1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity is the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high-scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence so that the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction is halted when the cumulative alignment score falls off by the quantity X from its maximum achieved value; the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4, and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, preferably less than about 0.01.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included in the Inactive Ingredient Guide prepared by the U.S. Food and Drug Administration.

The term “subject” or “host” refers to any individual who is the target of administration or treatment. The subject can be a vertebrate, for example, a mammal. Thus, the subject can be a human or veterinary patient. The term “patient” refers to a subject under the treatment of a clinician, e.g., physician. The subject can be either male or female.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

The term “tissue” refers to a group or layer of similarly specialized cells which together perform certain special functions. The term “tissue” is intended to include, blood, blood preparations such as plasma and serum, bones, joints, muscles, smooth muscles, lung tissues, and organs.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder (e.g., a cancer), or a symptom of a disease or disorder. The terms “treating” and “treatment” can also refer to reduction in severity and/or frequency of symptoms, elimination of symptoms and/or underlying cause, and/or improvement or remediation of damage.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition (e.g. cancer). Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

The term “colorectal cancer” includes cancer of the colon, rectum, and/or anus, and especially, adenocarcinomas, and may also include carcinomas (e.g., squamous cloacogenic carcinomas), melanomas, lymphomas, and sarcomas. Epidermoid (nonkeratinizing squamous cell or basaloid) carcinomas are also included. The cancer may be associated with particular types of polyps or other lesions, for example, tubular adenomas, tubulovillous adenomas (e.g., villoglandular polyps), villous (e.g., papillary) adenomas (with or without adenocarcinoma), hyperplastic polyps, hamartomas, juvenile polyps, polypoid carcinomas, pseudopolyps, lipomas, or leiomyomas. The cancer may be associated with familial polyposis and related conditions such as Gardner's syndrome or Peutz-Jeghers syndrome.

The cancer may be associated, for example, with chronic fistulas, irradiated anal skin, leukoplakia, lymphogranuloma venereum, Bowen's disease (intraepithelial carcinoma), condyloma acuminatum, or human papillomavirus. In other aspects, the cancer may be associated with basal cell carcinoma, extramammary Paget's disease, cloacogenic carcinoma, or malignant melanoma.

The terms “differentially expressed gene,” “differential gene expression,” and like phrases, refer to a gene whose expression is activated to a higher or lower level in a subject (e.g., test sample), specifically cancer, such as gastrointestinal cancer, relative to its expression in a control subject (e.g., control sample). The terms also include genes whose expression is activated to a higher or lower level at different stages of the same disease; in recurrent or non-recurrent disease; or in cells with higher or lower levels of proliferation. A differentially expressed gene may be either activated or inhibited at the polynucleotide level or polypeptide level, or may be subject to alternative splicing to result in a different polypeptide product. Such differences may be evidenced by a change in mRNA levels, surface expression, secretion or other partitioning of a polypeptide, for example.

Differential gene expression may include a comparison of expression between two or more genes or their gene products; or a comparison of the ratios of the expression between two or more genes or their gene products; or a comparison of two differently processed products of the same gene, which differ between normal subjects and diseased subjects; or between various stages of the same disease; or between recurring and non-recurring disease; or between cells with higher and lower levels of proliferation; or between normal tissue and diseased tissue, specifically cancer, or gastrointestinal cancer. Differential expressions include both quantitative, as well as qualitative, differences in the temporal or cellular expression pattern in a gene or its expression products among, for example, normal and diseased cells, or among cells which have undergone different disease events or disease stages, or cells with different levels of proliferation.

The term “expression” includes production of polynucleotides and polypeptides, in particular, the production of RNA (e.g., mRNA) from a gene or portion of a gene, and includes the production of a protein encoded by an RNA or gene or portion of a gene, and the appearance of a detectable material associated with expression. For example, the formation of a complex, for example, from a protein-protein interaction, protein-nucleotide interaction, or the like, is included within the scope of the term “expression”. Another example is the binding of a binding ligand, such as a hybridization probe or antibody, to a gene or other oligonucleotide, a protein or a protein fragment and the visualization of the binding ligand. Thus, increased intensity of a spot on a microarray, on a hybridization blot such as a Northern blot, or on an immunoblot such as a Western blot, or on a bead array, or by PCR analysis, is included within the term “expression” of the underlying biological molecule.

The term “gastric cancer” includes cancer of the stomach and surrounding tissue, especially adenocarcinomas, and may also include lymphomas and leiomyosarcomas. The cancer may be associated with gastric ulcers or gastric polyps, and may be classified as protruding, penetrating, spreading, or any combination of these categories, or, alternatively, classified as superficial (elevated, flat, or depressed) or excavated.

The term “recurrence free survival” is used herein to refer to survival without recurrence of CRC for at least 5 years, more preferably for at least 8 years, most preferably for at least 10 years following surgery or other treatment The term “prognosis” refers to a prediction of medical outcome (e.g., likelihood of long-term survival); a negative prognosis, or bad outcome, includes a prediction of relapse, disease progression (e.g., tumor growth or metastasis, or drug resistance), or mortality; a positive prognosis, or good outcome, includes a prediction of disease remission, (e.g., disease-free status), amelioration (e.g., tumor regression), or stabilization.

The terms “prognostic signature,” “signature,” and the like refer to a set of two or more markers, for example 10-gene TME^{S} and 10-gene EPI^{S} signatures, that when analyzed together as a set allow for the determination of or prediction of an event, for example the prognostic outcome of colorectal cancer. The use of a signature comprising two or more markers reduces the effect of individual variation and allows for a more robust prediction. Non-limiting examples of a 10-gene tumor microenvironment signature (TME^{S}), include, but are not limited to, the specific group comprising CD109, AHNAK2, GAS1, PRKCDBP, MEIS2, NXN, GFPT2, PMP22, WWTR1, or PTRF gene. Non-limiting examples of a 10-gene epithelial-associated signature (EPI^{S}), include, but are not limited to, the specific group comprising CDX1, CDX2, C10orf99, DDC, GPA33, FAM84A, NR1I2, MYB, C2orf89, or EPHB2 gene.

In the context of the present invention, reference to “at least one,” “at least two,” “at least five,” etc., of the markers listed in any particular set (e.g., any signature) means any one or any and all combinations of the markers listed.

The term “prediction method” is defined to cover the broader genus of methods from the fields of statistics, machine learning, artificial intelligence, and data mining, which can be used to specify a prediction model.

The term “prediction model” refers to the specific mathematical model obtained by applying a prediction method to a collection of data. In the examples, disclosed herein, such data sets consist of measurements of gene activity in tissue samples taken from recurrent and non-recurrent colorectal cancer patients, for which the class (recurrent or non-recurrent) of each sample is known. Such models can be used to (1) classify a sample of unknown recurrence status as being one of recurrent or non-recurrent, or (2) make a probabilistic prediction (i.e., produce either a proportion or percentage to be interpreted as a probability) which represents the likelihood that the unknown sample is recurrent, based on the measurement of mRNA expression levels or expression products, of a specified collection of genes, in the unknown sample. The exact details of how these gene-specific measurements are combined to produce classifications and probabilistic predictions are dependent on the specific mechanisms of the prediction method used to construct the model.

The term “proliferation” refers to the processes leading to increased cell size or cell number, and can include one or more of: tumor or cell growth, angiogenesis, innervation, and metastasis.

The term “qPCR” or “QPCR” refers to a quantitative polymerase chain reaction as described, for example, in PCR Technique: Quantitative PCR, J. W. Larrick, ed., Eaton Publishing, 1997, and A-Z of Quantitative PCR, S. Bustin, ed., IUL Press, 2004.

The term “tumor” refers to all neoplastic cell growth and proliferation, whether malignant or benign and all pre-cancerous and cancerous cells and tissues.

Sensitivity”, “specificity” (or “selectivity”), and “classification rate”, when applied to the describing the effectiveness of prediction models mean the following: “Sensitivity” means the proportion of truly positive samples that are also predicted (by the model) to be positive. In a test for cancer recurrence, that would be the proportion of recurrent tumors predicted by the model to be recurrent. “Specificity” or “selectivity” means the proportion of truly negative samples that are also predicted (by the model) to be negative. In a test for CRC recurrence, this equates to the proportion of non-recurrent samples that are predicted to by non-recurrent by the model. “Classification Rate” is the proportion of all samples that are correctly classified by the prediction model (be that as positive or negative).

The practice of the present invention will employ unless otherwise indicated, conventional techniques of molecular biology (including recombinant techniques), microbiology, cell biology, and biochemistry, which are within the skill of the art. Such techniques are explained fully in the literature, such as Molecular Cloning: A Laboratory Manual, 2nd edition, Sambrook et al., 1989; Oligonucleotide Synthesis, M J Gait, ed., 1984; Animal Cell Culture, R. I. Freshney, ed., 1987; Methods in Enzymology, Academic Press, Inc.; Handbook of Experimental Immunology, 4th edition, D. M. Weir & C C. Blackwell, eds., Blackwell Science Inc., 1987; Gene Transfer Vectors for Mammalian Cells, J. M. Miller & M. P. Calos, eds., 1987; Current Protocols in Molecular Biology, F. M. Ausubel et al., eds., 1987; and PCR: The Polymerase Chain Reaction, Mullis et al., eds., 1994.

In some examples, numbers expressing quantities of ingredients, properties such as molecular weight, reaction conditions, and so forth, used to describe and claim certain examples of the present disclosure are to be understood as being modified in some instances by the term “about.” In some examples, the term “about” is used to indicate that a value includes the standard deviation of the mean for the device or method being employed to determine the value. In some examples, the numerical parameters set forth in the written description and attached claims are approximations that can vary depending upon the desired properties sought to be obtained by a particular embodiment. In some examples, the numerical parameters should be construed in light of the number of reported significant digits and by applying ordinary rounding techniques. Notwithstanding that the numerical ranges and parameters setting forth the broad scope of some examples of the present disclosure are approximations, the numerical values set forth in the specific examples are reported as precisely as practicable. The numerical values presented in some examples of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

The evidence presented in this application, clearly demonstrates and quantifies the distinct cellular contributions of the tumor epithelium (EPI) vs. the tumor microenvironment (TME) in determining CRC prognosis and therapeutic outcomes.

An increase in TME^{S} score, for example, markers associated with stromal or immune cells in tumor microenvironment, is indicative of worse survival and resistance to CRC targeted therapy immunotherapy and MEK inhibitors. This can include decreased likelihood of cancer recurrence after standard treatment, especially for gastrointestinal cancer, such as gastric or colorectal cancer. Conversely, a decrease in these markers is indicative of a progression free survival (PFS), better overall survival (OS) and better relapse free survival (RFS). This can include disease progression or the increased likelihood of cancer recurrence, especially for gastrointestinal cancer, such as gastric or colorectal cancer. A decrease in expression can be determined, for example, by comparison of a biological sample from the CRC subject (e.g., tumor sample) to samples associated with a healthy subject (non-tumor) or subjects with low expression of TME^{S}/EPI^{S} (control). An increase in expression can be determined, for example, by comparison of a tumor sample to samples associated with a control/healthy subject.

As used herein, the “TME^{S} (Tumor Microenvironment Signature) score” and “EPI^{S}(Epithelial-Associated Signature) score” in colorectal cancer (CRC) datasets were calculated using a standardized bioinformatic approach based on gene expression data. Specifically, whole transcriptome sequencing (WTS) or Affymetrix gene expression data were utilized to quantify gene expression levels. The TME^{S}/EPI^{S} genes were first log-transformed and z-scored, ensuring normalization across samples. The TME^{S}/EPI^{S} signature scores were then computed as the arithmetic mean of the z-scores for all genes within the respective 10-gene signatures for a given specimen. This method was consistently applied across multiple CRC datasets, including the Total Cancer Care (TCC) dataset, CARIS LS real-world clinico-genomics dataset, TCGA CRC dataset, and Marisa et al. CRC dataset, to assess gene expression correlations with key driver mutations (e.g., BRAF, KRAS, APC, TP53) and clinical outcomes. Additionally, in clinical trial datasets (e.g., Merck PN04 and BMS Khambata-Ford), these scores were used to predict progression-free survival (PFS) and resistance to therapies such as cetuximab and panitumumab. In single-cell RNA sequencing (scRNA-seq) analyses, such as the Pelka et al. CRC dataset, TME^{S} and EPI^{S} expression profiles were assessed at a single-cell resolution to distinguish epithelial tumor cells from immune/stromal components. The scores were further validated in various public CRC datasets by correlating them with CMS classifications, MEK pathway activation, and EMT-related gene expression profiles, supporting their role in CRC stratification, prognosis, and treatment selection.

For example, to obtain a prognosis, a patient's sample (e.g., tumor sample) can be compared to samples with known patient outcome. If the patient's sample shows increased expression of TME^{S} score that is comparable to samples from healthy subject, and/or higher than samples with low expression of TME^{S}/EPI^{S}, then a worse survival and resistance to CRC targeted therapy immunotherapy and MEK inhibitors is implicated. If the patient's sample shows increase in EPI^{S} score that indicates better survival and sensitivity to CRC targeted therapies. Alternatively, a patient's sample can be compared to samples of actively proliferating/non-proliferating tumor cells. If the patient's sample shows increased expression of TME^{S} that is comparable to actively proliferating cells, and/or higher than non-proliferating cells in the tumor microenvironment, then a worse survival is implicated. If the patient's sample shows increased expression of EPI^{S} that is comparable to non-proliferating cells, and/or higher in actively proliferating epithelial cells, then a better overall survival is implicated.

In some examples, disclosed herein is a method of determining, calculating, computing, identifying, detecting, measuring, evaluating, assessing, deriving, and/or ascertaining CRC related therapy response, comprising obtaining a biological sample from a CRC subject, measuring expression levels of a 10-gene tumor microenvironment signature (TME^{S}), wherein the 10-gene tumor microenvironment signature comprises CD109, AHNAK2 (AHNAK nucleoprotein 2), GAS1 (Growth arrest-specific 1), PRKCDBP (Protein kinase C delta binding protein/CAVIN-3), MEIS2 (Meis homeobox 2), NXN (nucleoredoxin), GFPT2 (Glutamine-Fructose-6-Phosphate Transaminase 2), PMP22 (Peripheral myelin protein 22), WWTR1 (WW Domain Containing Transcription Regulator 1), or PTRF (Polymerase I and Transcript Release Factor/CAVIN-1) gene, a 10-gene epithelial signature (EPI^{S}), wherein the 10-gene epithelial signature comprises CDX1(Caudal Type Homeobox 1), CDX2 (Caudal Type Homeobox 2), C10orf99/CSBF (chromosome 10 open reading frame 99/SUSD2 binding factor), DDC (1-DOPA decarboxylase), GPA33 (Glycoprotein A33), FAM84A (family with sequence similarity 84, member A/LRATD1), NR1I2/PXR (pregnane X receptor), MYB (MYB Proto-Oncogene), C2orf89 (Chromosome 2 Open Reading Frame 89/TRABD2A), or EPHB2 (Ephrin Type-B Receptor 2) gene, or a combination thereof in the biological sample, calculating a TME^{S} score, EPI^{S} score or combination thereof, classifying the CRC subject based on the TME^{S} score or the EPI^{S} score, wherein a high TME^{S} score indicates worse survival and resistance to CRC targeted therapy immunotherapy and MEK inhibitors, wherein a high EPI^{S} score indicates better survival and sensitivity to CRC targeted therapies and administering a therapeutically effective dose of CRC targeted therapy (such as, for example, including but not limited to immunotherapy, Epidermal Growth Factor Receptor (EGFR) inhibitors, SRC inhibitors or MEK inhibitors) to the CRC subject.

In some examples, disclosed herein the CRC subject is classified into at least one of consensus molecular subtypes (CMS) consisting of a CMS1 subtype, a CMS2 subtype, a CMS3 subtype, and a CMS4 subtype. In some examples, at least CMS1 subtype is characterized by microsatellite instability (MSI) or immune activation, with worse survival after relapse (SAR), at least CMS2 subtype(canonical) is characterized by epithelial cell origin or WNT/MYC signaling, at least CMS3 subtype (metabolic) is characterized by epithelial origin or dysregulated metabolism, and at least CMS4 subtype (mesenchymal) is characterized by stromal infiltration, angiogenesis and TGFP activation in association with worse overall survival (OS) and worse relapse free survival (RFS).

In some examples, the CMS1 subtype or the CMS4 subtype is correlated to worse survival in the CRC subject. In some examples, disclosed herein the CMS1 subtype and the CMS4 subtype is correlated with the TME^{S} score. Also disclosed herein, the CMS1 subtype of any preceding aspect is positively correlated with memory B cells, CD8+ T cells, gamma delta T cells, NK cells, macrophages or dendritic cells. Disclosed herein, the CMS4 subtype of any preceding aspect is positively correlated with stromal cells or tumor cells.

In some examples, the CMS2 subtype or the CMS3 subtype is correlated to better survival in the CRC subject. In some examples, disclosed herein the CMS2 subtype and the CMS3 subtype are correlated with the EPI^{S} score. Also disclosed herein, the CMS2 subtype and the CMS3 subtype of any preceding aspect are correlated with inactive B cells, resting NK cells and macrophages (M0).

In some examples, the expression of a panel of markers in the 10-gene TME^{S} and 10-gene EPI^{S} can be analyzed by techniques including single-cell RNA sequencing (scRNA-seq). scRNA-seq leads to work out a TME^{S} based score or an EPI^{S} based score. The marker panel selected and prognostic score calculation can be derived through extensive laboratory testing and multiple independent clinical development studies. The score related gene expression signatures, such as TME^{S} and EPI^{S}, is typically derived from normalized gene expression data obtained from RNA sequencing or microarray analysis. The expression values are often normalized using methods like TPM (Transcripts Per Million), FPKM (Fragments Per Kilobase of transcript per Million mapped reads), or log 2-transformed expression values. A signature score can be calculated by taking the mean expression of all genes within the signature, assuming equal contribution from each gene. Alternatively, single-sample Gene Set Enrichment Analysis (ssGSEA) can be used to compute an enrichment score based on gene rank order, or Z-score normalization can be applied to standardize expression across samples. Another approach involves Principal Component Analysis (PCA), where the first principal component (PC1) is used as the signature score, capturing the most significant variation. Once these scores are computed, they are utilized in statistical analyses, including Cox regression models, to assess their prognostic significance in colorectal cancer. In the application, TME^{S} and EPI^{S} scores were analyzed alongside the 29-tumor microenvironment functional gene expression signatures (described in Bagaev et al.) to determine their independence as prognostic markers. The findings revealed that TME^{S} was particularly influenced by the Cancer-Associated Fibroblast (CAF) signature, as well as other immune and stromal signatures, demonstrating its ability to capture key tumor microenvironment components. In some examples, the TME^{S} score, and the EPI^{S} score is calculated by quantifying gene expression levels of 10-gene TME^{S} and 10-gene EPI^{S} signatures, respectively.

In some examples, the disclosed TME^{S}/EPI^{S} scores therefore provide a useful tool for determining the prognosis of cancer, and establishing a treatment regime specific to that tumor. In particular, a high EPI^{S} score prognosis can be used by a patient to decide to pursue standard or less invasive treatment options. A high TME^{S} score can be used by a patient to decide to terminate treatment or to pursue highly aggressive or experimental treatments. In addition, a patient can choose treatments based on their impact on cell proliferation or the expression of 10-gene TME^{S} in stromal or immune cells in tumor microenvironment vs. 10-gene EPI^{S} in epithelial tumor cells or epithelial normal mucosa. In accordance with the present invention, treatments that specifically target CRC subjects with low expression of 10-gene TME^{S} or high expression of 10-gene EPI^{S} would be preferred for administering a therapeutically effective dose of CRC targeted therapy (such as, for example, including but not limited to immunotherapy, Epidermal Growth Factor Receptor (EGFR) inhibitors, SRC inhibitors or MEK inhibitors).

In some examples, the immunotherapy agents comprise such as, for example, including but not limited to pembrolizumab (KEYTRUDA®), nivolumab (OPDIVO®), ipilimumab (YERVOY®), atezolizumab (TECENTRIQ®), or dostarlimab (JEMPERLI™), primarily targeting immune checkpoints like PD-1, PD-L1, or CTLA-4, offering effective treatment for microsatellite instability-high (MSI-H) or mismatch repair-deficient (dMMR) CRC cases. In some examples, the cancer-associated fibroblast (CAF)-related therapy comprises such as, for example, including but not limited fibroblast activation protein (FAP) inhibitors (such as, for example, talabostat), TGFβ inhibitors (such as, for example, galunisertib), CXCL12/CXCR4 inhibitors (such as, for example, plerixafor). Disclosed herein, in some examples administering a therapeutically effective amount of immunotherapy (checkpoint inhibitor therapy), SRC inhibitor therapy or cancer-associated fibroblast (CAF)-related therapy to a subject with high TME^{S} score.

In some examples, the EGFR inhibitors comprise such as, for example, including but not limited to cetuximab (ERBITUX®) or panitumumab (VECTIBIX®), blocking EGFR signaling and are used in patients with RAS wild-type tumors. In some examples, the MEK inhibitors comprise such as, for example, including but not limited to trametinib (MEKINIST®), binimetinib (MEKTOVI®), or selumetinib (KOSELUGO®), acting on the MAPK/ERK signaling pathway, offering benefits in CRC cases with mutations in the RAS-RAF pathway. These agents are used either as monotherapies or in combination with other treatments to enhance efficacy and improve patient outcomes. Disclosed herein, in some examples administering a therapeutically effective amount of MEK inhibitors or EGFR inhibitors to a subject with high EPI^{S} score.

In some examples, gene expression levels of 10-gene TME^{S} and 10-gene EPI^{S} can be detected in tumor tissue, tissue proximal to the tumor, a surgical resection specimen, tissue biopsy (such as, for example, tissue section, fixed sample, formalin fixed, paraffin embedded (FFPE) sample), fine needle aspirate, lymph node samples, blood samples, serum samples, urine samples, or fecal samples, using any suitable technique, and can include, but is not limited to, oligonucleotide probes, quantitative PCR, or antibodies raised against the signatures. A high expression level of at least one gene in the 10-gene TME^{S} biomarker panel in the sample will be indicative of the likelihood of recurrence in that subject. However, it will be appreciated that by analyzing the presence and amounts of expression of a plurality of all the 10-genes in the biomarker panels (TME^{S} and EPI^{S}), and constructing a TME^{S} score or an EPI^{S} score, the sensitivity and accuracy of prognosis will be increased. Therefore, multiple markers according to the present invention can be used to determine the prognosis and therapeutic outcomes of CRC. The disclosed method includes the use of archived paraffin-embedded biopsy material for assay of all biomarkers in the TME^{S} and EPI^{S}, and therefore is compatible with the most widely available type of biopsy material. It is also compatible with several different methods of tumor tissue harvest, for example, via core biopsy or fine needle aspiration. In a further aspect, RNA is isolated from a fixed, wax-embedded cancer tissue specimen of the patient. Isolation may be performed by any technique known in the art, for example from core biopsy tissue or fine needle aspirate cells.

The present invention relates to a set of markers, in particular, the 10-gene TME^{S} versus the 10-gene EPI^{S} signature scores, wherein the biomarkers are capable of quantifying the dependency of clinical outcomes on tumor epithelial cells vs. the TME, which may ultimately help optimize therapeutic strategies for CRC patients. For example, the high expression of EPI^{S} biomarkers have a prognostic value, specifically with respect to relapse free survival. In specific aspects, the cancer is gastrointestinal cancer, particularly, gastric or colorectal cancer.

In some examples, disclosed herein is a method for treating, inhibiting, reducing, decreasing, ameliorating, and/or preventing CRC in a subject, comprising obtaining a biological sample from the subject, measuring expression levels of a 10-gene tumor microenvironment signature (TME^{S}), wherein the TME^{S} comprises CD109, AHNAK2, GAS1, PRKCDBP, MEIS2, NXN, GFPT2, PMP22, WWTR1, or PTRF gene, calculating a TME^{S} score, analyzing presence of CRC subtype CMS4 or subtype CMS1 based on a high TME^{S} score, and administering a therapeutically effective amount of immunotherapy (checkpoint inhibitor therapy), SRC inhibitor therapy or cancer-associated fibroblast (CAF)-related therapy to the subject. In some examples, the expression levels of one or more tumor microenvironment signature (TME^{S}), for example at least two, or at least 3, or at least 4, or at least 5, or at least 10 of the TME signature biomarkers or their expression products are determined, e.g., as selected from CD109, AHNAK2, GAS1, PRKCDBP (CAVIN3), MEIS2, NXN, GFPT2, PMP22, WWTR1 or PTRF (CAVIN1).

As used herein the “Cancer-Associated Fibroblast (CAF)-related therapy” in colorectal cancer includes cancer-associated fibroblasts (CAFs) which play a crucial role in the tumor microenvironment (TME) by promoting tumor growth, invasion, metastasis, immune evasion, and resistance to therapy. Targeting CAFs is a promising strategy in colorectal cancer (CRC) treatment. Several approaches are being explored to modulate or inhibit CAF activity, including CAF depletion strategies (such as, for example, including but not limited to fibroblast activation protein (FAP) inhibitors (e.g. Talabostat), anti-FAP antibody-drug conjugates (ADCs) (e.g. FAP-TAK-228 ADCs), reprogramming CAFs to anti-tumorigenic states, TGF-β inhibitors (e.g. Galunisertib) or CXCL12/CXCR4 inhibitors (e.g. Plerixafor®).

In some examples, disclosed herein is a method for treating, inhibiting, reducing, decreasing, ameliorating, and/or preventing CRC in a subject, comprising obtaining a biological sample from the subject, measuring expression levels of 10-gene epithelial-associated signature (EPI^{S}), wherein the EPI^{S} comprises CDX1, CDX2, C10orf99, DDC, GPA33, FAM84A, NR1I2, MYB, C2orf89, or EPHB2 gene, calculating an EPI^{S} score, analyzing presence of CRC subtype CMS2 or subtype CMS3 based on a high EPI^{S} score, and administering a therapeutically effective amount of EGFR inhibitor (EGFRi) therapy to the subject.

In some examples, the expression levels of one or more epithelial-associated signature (EPI^{S}), for example at least two, or at least 3, or at least 4, or at least 5, or at least 10 of the EPI signature biomarkers or their expression products are determined, e.g., as selected from CDX1, CDX2, C10orf99, DDC, GPA33, FAM84A (LRATD1), NR1I2, MYB, C2orf89 (TRABD2A) or EPHB2.

In some examples, the invention relates to a method of predicting the likelihood of long-term survival of a patient diagnosed with cancer, without the recurrence of cancer, comprising the steps of: (1) determining the expression levels of the RNA transcripts or the expression products of the full set or a subset of the biomarker panel listed in 10-gene epithelial-associated signature (EPI^{S}), or 10-gene tumor microenvironment signature (TME^{S}), herein, in a sample obtained from the patient, normalized against the expression levels of all RNA transcripts or their expression products in the sample, or of a reference set of RNA transcripts or their products; (2) subjecting the data obtained in step (1) to statistical analysis; and (3) determining whether the likelihood of better overall survival has increased or decreased.

In some examples, the invention concerns a method of stratifying, classifying, segmenting, categorizing, grouping, dividing, differentiating, and/or sorting a subject with CRC for targeted therapy selection, comprising obtaining a biological sample from the subject, performing single-cell RNA sequencing (scRNA-seq) analysis on the biological sample, identifying TME^{S}-positive or EPI^{S}-positive cells based on gene expression signatures, calculating a TME^{S} score or an EPI^{S} score, administering a therapeutically effective amount of immunotherapy, SRC inhibitor therapy or cancer-associated fibroblast (CAF)-related therapy to the subject with high TME^{S} score, and administering a therapeutically effective amount of EGFRi therapy or MEK inhibitor therapy to the subject with high EPI^{S} score.

In some examples, the invention relates to a prognostic method comprising: (a) subjecting a sample obtained from a patient to quantitative analysis of the expression level of the RNA transcript of at least one biomarker selected from the TME^{S} or the EPI^{S}, and (b) identifying the patient as likely to have an increased likelihood of relapse free survival without cancer recurrence if the normalized expression levels of the EPI^{S} biomarker or biomarkers, or their products, are above defined expression threshold. In alternate aspects, step (b) comprises identifying the patient as likely to have a decreased likelihood of overall survival without cancer recurrence if the normalized expression levels of the TME^{S} biomarker or biomarkers, or their products, are increased above a defined expression threshold.

The disclosed combined biomarker panel in a CRC subject therefore provide a useful set of biomarkers to generate prediction signatures for determining the prognosis of cancer, and establishing a treatment regime, or treatment modality, specific for that tumor. In particular, a 10-gene epithelial-associated signature (EPI^{S}), wherein the EPI^{S} comprises CDX1, CDX2, C10orf99, DDC, GPA33, FAM84A (LRATD1), NR1I2, MYB, C2orf89 (TRABD2A) or EPHB2 genes, and a 10-gene tumor microenvironment signature (TME^{S}), wherein the TME^{S} comprises CD109, AHNAK2, GAS1, PRKCDBP (CAVIN3), MEIS2, NXN, GFPT2, PMP22, WWTR1 or PTRF (CAVIN1)genes, wherein gene expression levels of EPI^{S} or TME^{S} are measured in a sample obtained from the CRC subject.

In some examples, the invention relates to a kit comprising one or more of: (1) extraction buffer/reagents and protocol; (2) reverse transcription buffer/reagents and protocol for 10-gene TME^{S} and/or 10-gene EPI^{S}; and (3) quantitative PCR buffer/reagents and protocol suitable for performing any of the foregoing methods. Other aspects and advantages of the invention are illustrated in the examples included herein.

It is understood and herein contemplated that the disclosed treatment regimens can used alone or in combination with any anti-cancer therapy known in the art including, but not limited to Abemaciclib, Abiraterone Acetate, Abitrexate® (Methotrexate), ABRAXANE® (Paclitaxel Albumin-stabilized Nanoparticle Formulation), ABVD, ABVE, ABVE-PC, AC, AC-T, ADCETRIS® (Brentuximab Vedotin), ADE, Ado-Trastuzumab Emtansine, Adriamycin® (Doxorubicin Hydrochloride), Afatinib Dimaleate, Afinitor® (Everolimus), Akynzeo® (Netupitant and Palonosetron Hydrochloride), ALDARA® (Imiquimod), Aldesleukin, ALECENSA® (Alectinib), Alectinib, Alemtuzumab, ALIMTA® (Pemetrexed Disodium), ALIQOPA® (Copanlisib Hydrochloride), ALKERAN™ for Injection (Melphalan Hydrochloride), ALKERAN™ Tablets (Melphalan), Aloxi® (Palonosetron Hydrochloride), Alunbrig® (Brigatinib), Ambochlorin® (Chlorambucil), Amboclorin® (Chlorambucil), Amifostine, Aminolevulinic Acid, Anastrozole, Aprepitant, Aredia® (Pamidronate Disodium), Arimidex® (Anastrozole), Aromasin® (Exemestane), Arranon® (Nelarabine), Arsenic Trioxide, Arzerra® (Ofatumumab), Asparaginase Erwinia chrysanthemi, Atezolizumab, Avastin® (Bevacizumab), Avelumab, Axitinib, Azacitidine, Bavencio® (Avelumab), BEACOPP, Becenum® (Carmustine), Beleodaq® (Belinostat), Belinostat, Bendamustine Hydrochloride, BEP, Besponsa® (Inotuzumab Ozogamicin), Bevacizumab, Bexarotene, Bexxar® (Tositumomab and Iodine I 131 Tositumomab), Bicalutamide, BiCNU® (Carmustine), Bleomycin, Blinatumomab, Blincyto® (Blinatumomab), Bortezomib, Bosulif® (Bosutinib), Bosutinib, Brentuximab Vedotin, Brigatinib, BuMel, Busulfan, Busulfex® (Busulfan), Cabazitaxel, Cabometyx® (Cabozantinib-S-Malate), Cabozantinib-S-Malate, CAF, Campath® (Alemtuzumab), Camptosar® (Irinotecan Hydrochloride), Capecitabine, CAPOX, Carac® (Fluorouracil—Topical), Carboplatin, CARBOPLATIN-TAXOL, Carfilzomib, Carmubris® (Carmustine), Carmustine, Carmustine Implant, Casodex® (Bicalutamide), CEM, Ceritinib, Cerubidine® (Daunorubicin Hydrochloride), Cervarix® (Recombinant HPV Bivalent Vaccine), Cetuximab, CEV, Chlorambucil, CHLORAMBUCIL-PREDNISONE, CHOP, Cisplatin, Cladribine, Clafen® (Cyclophosphamide), Clofarabine, Clofarex® (Clofarabine), Clolar® (Clofarabine), CMF, Cobimetinib, Cometriq® (Cabozantinib-S-Malate), Copanlisib Hydrochloride, COPDAC, COPP, COPP-ABV, Cosmegen® (Dactinomycin), Cotellic® (Cobimetinib), Crizotinib, CVP, Cyclophosphamide, Cyfos® (Ifosfamide), Cyramza® (Ramucirumab), Cytarabine, Cytarabine Liposome, Cytosar-U® (Cytarabine), Cytoxan® (Cyclophosphamide), Dabrafenib, Dacarbazine, Dacogen® (Decitabine), Dactinomycin, Daratumumab, Darzalex® (Daratumumab), Dasatinib, Daunorubicin Hydrochloride, Daunorubicin Hydrochloride and Cytarabine Liposome, Decitabine, Defibrotide Sodium, Defitelio® (Defibrotide Sodium), Degarelix, Denileukin Diftitox, Denosumab, DepoCyt® (Cytarabine Liposome), Dexamethasone, Dexrazoxane Hydrochloride, Dinutuximab, Docetaxel, Doxil® (Doxorubicin Hydrochloride Liposome), Doxorubicin Hydrochloride, Doxorubicin Hydrochloride Liposome, Dox-SL® (Doxorubicin Hydrochloride Liposome), DTIC-Dome® (Dacarbazine), Durvalumab, Efudex® (Fluorouracil—Topical), Elitek® (Rasburicase), Ellence® (Epirubicin Hydrochloride), Elotuzumab, Eloxatin® (Oxaliplatin), Eltrombopag Olamine, Emend® (Aprepitant), Empliciti® (Elotuzumab), Enasidenib Mesylate, Enzalutamide, Epirubicin Hydrochloride, EPOCH, Erbitux® (Cetuximab), Eribulin Mesylate, Erivedge® (Vismodegib), Erlotinib Hydrochloride, Erwinaze® (Asparaginase Erwinia chrysanthemi), Ethyol® (Amifostine), Etopophos Etopophos® (Etoposide Phosphate), Etoposide, Etoposide Phosphate, Evacet® (Doxorubicin Hydrochloride Liposome), Everolimus, Evista® (Raloxifene Hydrochloride), Evomela® (Melphalan Hydrochloride), Exemestane, 5-FU® (Fluorouracil Injection), 5-FU® (Fluorouracil—Topical), Fareston® (Toremifene), Farydak® (Panobinostat), Faslodex® (Fulvestrant), FEC, Femara® (Letrozole), Filgrastim, Fludara® (Fludarabine Phosphate), Fludarabine Phosphate, Fluoroplex® (Fluorouracil—Topical), Fluorouracil Injection, Fluorouracil—Topical, Flutamide, Folex® (Methotrexate), Folex PFS® (Methotrexate), FOLFIRI, FOLFIRI-BEVACIZUMAB, FOLFIRI-CETUXIMAB, FOLFIRINOX, FOLFOX, Folotyn® (Pralatrexate), FU-LV, Fulvestrant, Gardasil® (Recombinant HPV Quadrivalent Vaccine), Gardasil 9® (Recombinant HPV Nonavalent Vaccine), Gazyva® (Obinutuzumab), Gefitinib, Gemcitabine Hydrochloride, GEMCITABINE-CISPLATIN, GEMCITABINE-OXALIPLATIN, Gemtuzumab Ozogamicin, Gemzar® (Gemcitabine Hydrochloride), Gilotrif® (Afatinib Dimaleate), Gleevec® (Imatinib Mesylate), Gliadel® (Carmustine Implant), Gliadel Wafer® (Carmustine Implant), Glucarpidase, Goserelin Acetate, Halaven® (Eribulin Mesylate), Hemangeol® (Propranolol Hydrochloride), Herceptin® (Trastuzumab), HPV Bivalent Vaccine, Recombinant, HPV Nonavalent Vaccine, Recombinant, HPV Quadrivalent Vaccine, Recombinant, Hycamtin® (Topotecan Hydrochloride), Hydrea® (Hydroxyurea), Hydroxyurea, Hyper-CVAD, Ibrance® (Palbociclib), Ibritumomab Tiuxetan, Ibrutinib, ICE, Iclusig® (Ponatinib Hydrochloride), Idamycin® (Idarubicin Hydrochloride), Idarubicin Hydrochloride, Idelalisib, Idhifa® (Enasidenib Mesylate), Ifex® (Ifosfamide), Ifosfamide, Ifosfamidum® (Ifosfamide), IL-2 (Aldesleukin), Imatinib Mesylate, Imbruvica® (Ibrutinib), Imfinzi® (Durvalumab), Imiquimod, Imlygic® (Talimogene Laherparepvec), Inlyta® (Axitinib), Inotuzumab Ozogamicin, Interferon Alfa-2b, Recombinant, Interleukin-2 (Aldesleukin), Intron A® (Recombinant Interferon Alfa-2b), Iodine I 131 Tositumomab and Tositumomab, Ipilimumab, Iressa® (Gefitinib), Irinotecan Hydrochloride, Irinotecan Hydrochloride Liposome, Istodax® (Romidepsin), Ixabepilone, Ixazomib Citrate, Ixempra® (Ixabepilone), Jakafi® (Ruxolitinib Phosphate), JEB, Jevtana® (Cabazitaxel), Kadcyla® (Ado-Trastuzumab Emtansine), Keoxifene® (Raloxifene Hydrochloride), Kepivance® (Palifermin), Keytruda® (Pembrolizumab), Kisqali® (Ribociclib), Kymriah® (Tisagenlecleucel), Kyprolis® (Carfilzomib), Lanreotide Acetate, Lapatinib Ditosylate, Lartruvo® (Olaratumab), Lenalidomide, Lenvatinib Mesylate, Lenvima® (Lenvatinib Mesylate), Letrozole, Leucovorin Calcium, Leukeran® (Chlorambucil), Leuprolide Acetate, Leustatin® (Cladribine), Levulan® (Aminolevulinic Acid), Linfolizin® (Chlorambucil), LipoDox® (Doxorubicin Hydrochloride Liposome), Lomustine, Lonsurf® (Trifluridine and Tipiracil Hydrochloride), Lupron® (Leuprolide Acetate), Lupron Depot® (Leuprolide Acetate), Lupron Depot-Ped® (Leuprolide Acetate), Lynparza® (Olaparib), Marqibo® (Vincristine Sulfate Liposome), Matulane® (Procarbazine Hydrochloride), Mechlorethamine Hydrochloride, Megestrol Acetate, Mekinist® (Trametinib), Melphalan, Melphalan Hydrochloride, Mercaptopurine, Mesna, Mesnex® (Mesna), Methazolastone® (Temozolomide), Methotrexate, Methotrexate LPF® (Methotrexate), Methylnaltrexone Bromide, Mexate® (Methotrexate), Mexate-AQ® (Methotrexate), Midostaurin, Mitomycin C, Mitoxantrone Hydrochloride, Mitozytrex® (Mitomycin C), MOPP, Mozobil® (Plerixafor), Mustargen® (Mechlorethamine Hydrochloride), Mutamycin® (Mitomycin C), Myleran® (Busulfan), Mylosar® (Azacitidine), Mylotarg® (Gemtuzumab Ozogamicin), Nanoparticle Paclitaxel® (Paclitaxel Albumin-stabilized Nanoparticle Formulation), Navelbine® (Vinorelbine Tartrate), Necitumumab, Nelarabine, Neosar® (Cyclophosphamide), Neratinib Maleate, Nerlynx® (Neratinib Maleate), Netupitant and Palonosetron Hydrochloride, Neulasta® (Pegfilgrastim), Neupogen® (Filgrastim), Nexavar® (Sorafenib Tosylate), Nilandron® (Nilutamide), Nilotinib, Nilutamide, Ninlaro® (Ixazomib Citrate), Niraparib Tosylate Monohydrate, Nivolumab, Nolvadex® (Tamoxifen Citrate), Nplate® (Romiplostim), Obinutuzumab, Odomzo® (Sonidegib), OEPA, Ofatumumab, OFF, Olaparib, Olaratumab, Omacetaxine Mepesuccinate, Oncaspar® (Pegaspargase), Ondansetron Hydrochloride, Onivyde® (Irinotecan Hydrochloride Liposome), Ontak® (Denileukin Diftitox), Opdivo® (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat® (Carboplatin), Paraplatin® (Carboplatin), Pazopanib Hydrochloride, PCV, PEB, Pegaspargase, Pegfilgrastim, Peginterferon Alfa-2b, PEG-Intron® (Peginterferon Alfa-2b), Pembrolizumab, Pemetrexed Disodium, Perjeta® (Pertuzumab), Pertuzumab, Platinol® (Cisplatin), Platinol-AQ® (Cisplatin), Plerixafor, Pomalidomide, Pomalyst® (Pomalidomide), Ponatinib Hydrochloride, Portrazza® (Necitumumab), Pralatrexate, Prednisone, Procarbazine Hydrochloride, Proleukin® (Aldesleukin), Prolia® (Denosumab), Promacta® (Eltrombopag Olamine), Propranolol Hydrochloride, Provenge® (Sipuleucel-T), Purinethol® (Mercaptopurine), Purixan® (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor® (Methylnaltrexone Bromide), R-EPOCH, Revlimid® (Lenalidomide), Rheumatrex® (Methotrexate), Ribociclib, R-ICE, Rituxan® (Rituximab), Rituxan Hycela® (Rituximab and Hyaluronidase Human), Rituximab, Rituximab and, Hyaluronidase Human, Rolapitant Hydrochloride, Romidepsin, Romiplostim, Rubidomycin® (Daunorubicin Hydrochloride), Rubraca® (Rucaparib Camsylate), Rucaparib Camsylate, Ruxolitinib Phosphate, Rydapt® (Midostaurin), Sclerosol Intrapleural Aerosol (Talc), Siltuximab, Sipuleucel-T, Somatuline Depot® (Lanreotide Acetate), Sonidegib, Sorafenib Tosylate, Sprycel® (Dasatinib), STANFORD V, Sterile Talc Powder (Talc), Steritalc® (Talc), Stivarga® (Regorafenib), Sunitinib Malate, Sutent® (Sunitinib Malate), Sylatron® (Peginterferon Alfa-2b), Sylvant® (Siltuximab), Synribo Synribo® (Omacetaxine Mepesuccinate), Tabloid® (Thioguanine), TAC, Tafinlar® (Dabrafenib), Tagrisso® (Osimertinib), Talc, Talimogene Laherparepvec, Tamoxifen Citrate, Tarabine PFS® (Cytarabine), Tarceva® (Erlotinib Hydrochloride), Targretin® (Bexarotene), Tasigna® (Nilotinib), Taxol® (Paclitaxel), Taxotere® (Docetaxel), Tecentriq® (Atezolizumab), Temodar® (Temozolomide), Temozolomide, Temsirolimus, Thalidomide, Thalomid® (Thalidomide), Thioguanine, Thiotepa, Tisagenlecleucel, Tolak® (Fluorouracil—Topical), Topotecan Hydrochloride, Toremifene, Torisel® (Temsirolimus), Tositumomab and Iodine I 131 Tositumomab, Totect® (Dexrazoxane Hydrochloride), TPF, Trabectedin, Trametinib, Trastuzumab, Treanda® (Bendamustine Hydrochloride), Trifluridine and Tipiracil Hydrochloride, Trisenox® (Arsenic Trioxide), Tykerb® (Lapatinib Ditosylate), Unituxin® (Dinutuximab), Uridine Triacetate, VAC, Vandetanib, VAMP, Varubi® (Rolapitant Hydrochloride), Vectibix® (Panitumumab), VeIP, Velban® (Vinblastine Sulfate), Velcade® (Bortezomib), Velsar® (Vinblastine Sulfate), Vemurafenib, Venclexta® (Venetoclax), Venetoclax, Verzenio® (Abemaciclib), Viadur® (Leuprolide Acetate), Vidaza® (Azacitidine), Vinblastine Sulfate, Vincasar PFS® (Vincristine Sulfate), Vincristine Sulfate, Vincristine Sulfate Liposome, Vinorelbine Tartrate, VIP, Vismodegib, Vistogard® (Uridine Triacetate), Voraxaze® (Glucarpidase), Vorinostat, Votrient® (Pazopanib Hydrochloride), Vyxeos® (Daunorubicin Hydrochloride and Cytarabine Liposome), Wellcovorin® (Leucovorin Calcium), Xalkori® (Crizotinib), Xeloda® (Capecitabine), XELIRI, XELOX, Xgeva® (Denosumab), Xofigo® (Radium 223 Dichloride), Xtandi® (Enzalutamide), Yervoy® (Ipilimumab), Yondelis® (Trabectedin), Zaltrap® (Ziv-Aflibercept), Zarxio® (Filgrastim), Zejula® (Niraparib Tosylate Monohydrate), Zelboraf® (Vemurafenib), Zevalin® (Ibritumomab Tiuxetan), Zinecard® (Dexrazoxane Hydrochloride), Ziv-Aflibercept, Zofran® (Ondansetron Hydrochloride), Zoladex® (Goserelin Acetate), Zoledronic Acid, Zolinza® (Vorinostat), Zometa® (Zoledronic Acid), Zydelig® (Idelalisib), Zykadia® (Ceritinib), and/or Zytiga® (Abiraterone Acetate). The treatment methods can include or further include checkpoint inhibitors including, but are not limited to antibodies that block PD-1 (such as, for example, Nivolumab (BMS-936558 or MDX1106), pembrolizumab, cemiplimab, CT-011, MK-3475), PD-L1 (such as, for example, atezoliziumab, avelumiab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, BI754111, Sym022, FS118, MGD013, and Immutep).

### EXAMPLES

The following examples are set forth below to illustrate the compounds, systems, methods, and results according to the disclosed subject matter. These examples are not intended to be inclusive of all examples of the subject matter disclosed herein, but rather to illustrate representative methods and results. These examples are not intended to exclude equivalents and variations of the present invention which are apparent to one skilled in the art. Unless defined otherwise, all technical and scientific terms used herein have the same meanings as commonly understood by one of skill in the art to which the disclosed invention belongs. Publications cited herein and the materials for which they are cited are specifically incorporated by reference.

### Example 1: The Highly Prognostic ΔPC1.EMT Score was Strongly Associated with CMS1 and CMS4 Subtypes Independent of Age, Sex, Race, Stages, MSI, Metastasis and Sidedness in 2373 CRCs

Merck-Moffitt 2373 CRC tumors were classified by the CMS subtypes using the CMScaller. Among 2373 tumor samples analyzed, CMS1-4 subtypes could be determined in 2120 samples whereas 253 tumors were not applicable to be classified into any CMS1-4 subtypes (CMS-NA). The frequencies of CMS subtypes were as follows: CMS1 (n=329; 14%), CMS2 (n=689, 29%), CMS3 (n=357; 15%), and CMS4 (n=745; 31%) as well as CMS-NA (n 00253; 11%). Various baseline phenotypic characteristics and related statistics among CMS subtypes are listed in Table 1. Here the p values based on standardized residual were used to compare the subgroups of the baseline phenotypic characteristics in each CMS subtype. The Cramer's V (last column) shows the strength of their association with the CMS classes. It is noteworthy that the baseline characteristics of CMS1 and CMS3 tumors were generally shared, as were the CMS2 and CMS4 tumors. Notably, among patients identified with “Race and Ethnicity” information, 700 of patients were “Black or African American” and appeared to have lower CMS1 tumors (4%, P<0.05).

TABLE 1
Baseline characteristics of Moffitt-Merck 2373 CRC tumors.

| | Total | CMS1 | CMS2 | CMS3 | CMS4 | CMS-NA | |
|---|---|---|---|---|---|---|---|
| | (n = 2373) | (n = 329) | (n = 689) | (n = 357) | (n = 745) | (n = 253) | Chi-Square |
| Characteristic | (100%) | (14%) | (29%) | (15%) | (31%) | (11%) | p-Value |
| Age (yr) | | | | | | | |
| Mean | 66 | 70 | 65 | 68 | 64 | 66 | |
| Median | 67 | 72 | 66 | 69 | 64 | 68 | |
| Range | (21-88) | (21-87) | (24-88) | (26-87) | (28-87) | (31-87) | |
| Age subgroups | 2373 (100%) | 329 (100%) | 689 (100%) | 357 (100%) | 745 (100%) | 253 (100%) | |
| (yr) | | | | | | | |
| <50 | 268 (11%) | 25 (8%) | 74 (11%) | 29 (8%) ↓ | 112 (15%) ↑↑↑ | 28 (11%) | <.0001 |
| 50-59 | 452 (19%) | 44 (13%) ↓↓ | 147 (21%) | 63 (18%) | 154 (20%) | 44 (17%) | V = .101 |
| 60-69 | 664 (28%) | 68 (21%) ↓↓ | 202 (29%) | 98 (27%) | 229 (31%) ↑ | 67 (26%) | |
| ≥70 | 989 (42%) | 192 (58%) ↑↑↑ | 266 (39%) | 167 (47%) ↑ | 250 (34%) ↓↓↓ | 114 (45%) | |
| Sex | 2372 (100%) | 329 (100%) | 688 (100%) | 357 (100%) | 745 (100%) | 253 (100%) | |
| Male | 1243 (52%) | 130 (40%) ↓↓↓ | 401 (58%) ↑↑↑ | 175 (49%) | 402 (54%) | 135 (53%) | <.0001 |
| Female | 1129 (48%) | 199 (60%) ↑↑↑ | 287 (42%) ↓↓↓ | 182 (51%) | 343 (46%) | 118 (47%) | V = .120 |
| Race | 2076 (100%) | 290 (100%) | 595 (100%) | 304 (100%) | 666 (100%) | 221 (100%) | |
| Asian | 12 (1%) | 1 (0%) | 3 (1%) | 1 (0 %) | 4 (1%) | 3 (1%) | .084ª |
| Black or African | 142 (7%) | 12 (4%) ↓ | 44 (7%) | 23 (8%) | 54 (8%) | 9 (4%) | V = .063ª |
| American | | | | | | | |
| White | 1900 (92%) | 273 (94%) ↑ | 542 (91%) | 278 (91%) | 599 (90%) | 208 (94%) | |
| Other | 22 (1%) | 4 (1%) | 6 (1%) | 2 (1%) | 9 (1%) | 1 (0%) | |
| Ethnicity | 1772 (100%) | 245 (100%) | 513 (100%) | 255 (100%) | 573 (100%) | 186 (100%) | |
| Hispanic or | 79 (4%) | 10 (4%) | 29 (6%) | 15 (6%) | 18 (3%) | 7 (4%) | .23 |
| Latino | | | | | | | |
| Not Hispanic or | 1693 (96%) | 235 (96%) | 484 (94%) | 240 (94%) | 555 (97%) | 179 (96%) | V = .056 |
| Latino | | | | | | | |
| Stage at | 2156 (100%) | 306 (100%) | 640 (100%) | 337 (100%) | 641 (100%) | 232 (100%) | |
| diagnosis | | | | | | | |
| 1 (0^{b}) | 278 (13%) | 40 (13%) | 85 (13%) | 86 (26%) ↑↑↑ | 43 (7%) ↓↓↓ | 24 (10%) | <.0001 |
| 2 | 491 (23%) | 111 (36%) ↑↑↑ | 130 (20%) | 79 (23%) | 138 (22%) | 33 (14%) ↓↓↓ | V = .158 |
| 3 | 579 (27%) | 73 (24%) | 145 (23%) ↓↓ | 107 (32%) ↑ | 187 (29%) | 67 (29%) | |
| 4 | 808 (37%) | 82 (27%) ↓↓↓ | 280 (4%) ↑↑↑ | 65 (19%) ↓↓↓ | 273 (43%) ↑↑ | 108 (47%) ↑↑ | |
| Microsatellite | 2373 (100%) | 329 (100%) | 689 (100%) | 357 (100%) | 745 (100%) | 253 (100%) | |
| status | | | | | | | |
| MSI | 237 (10%) | 165 (50%) ↑↑↑ | 0 (0%) ↓↓↓ | 41 (11%) | 12 (2%) ↓↓↓ | 19 (8%) | <.0001 |
| MSS | 2136 (90%) | 164 (50%) ↓↓↓ | 689 (100%) ↑↑↑ | 316 (89%) | 733 (98%) ↑↑↑ | 234 (92%) | V = .554 |
| Specimen type | 2373 (100%) | 329 (100%) | 689 (100%) | 357 (100%) | 745 (100%) | 253 (100%) | |
| Primary | 1571 (66%) | 256 (78%) ↑↑↑ | 412 (60%) ↓↓↓ | 318 (89%) ↑↑↑ | 448 (60%) ↓↓↓ | 137 (54%) ↓↓↓ | <.0001 |
| Metastatic | 802 (34%) | 73 (22%) ↓↓↓ | 277 (40%) ↑↑↑ | 39 (11%) ↓↓↓ | 297 (40%) ↑↑↑ | 116 (46%) ↑↑↑ | V = .247 |
| Sidedness | 1526 (100%) | 243 (100%) | 410 (100%) | 296 (100%) | 435 (100%) | 142 (100%) | |
| Left | 826 (54%) | 54 (22%)↓↓↓ | 300 (73%) ↑↑↑ | 132 (45%) ↓↓↓ | 275 (63%) ↑↑↑ | 65 (46%) ↓ | <.0001 |
| Right | 700 (46%) | 189 (78%) ↑↑↑ | 110 (27%)↓↓↓ | 164 (55%) ↑↑↑ | 160 (37%) ↓↓↓ | 77 (54%) ↑ | V = .352 |

Notes:
ªcomparing Black with White;
^{b}including 6 Stage 0 cases; the CMS subtypes were generated by the CMScaller.
↑(↓) for .01 < p < .05; ↑↑(↓↓) for .001 < p < .01; ↑↑↑(↓↓↓) for p < .001 based on the standardized residual; Cramer's V (last column) shows the strength of the association.

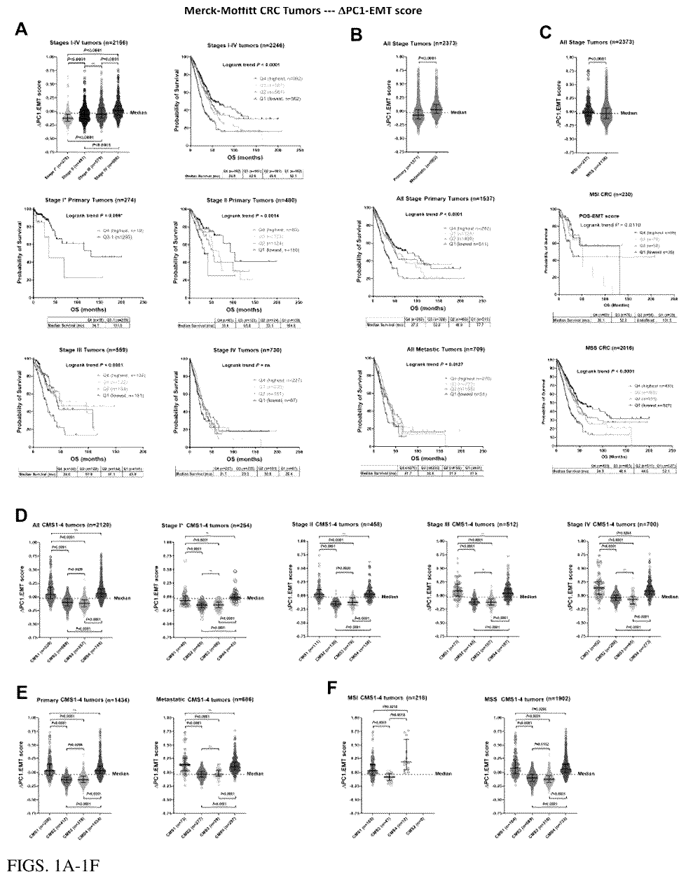

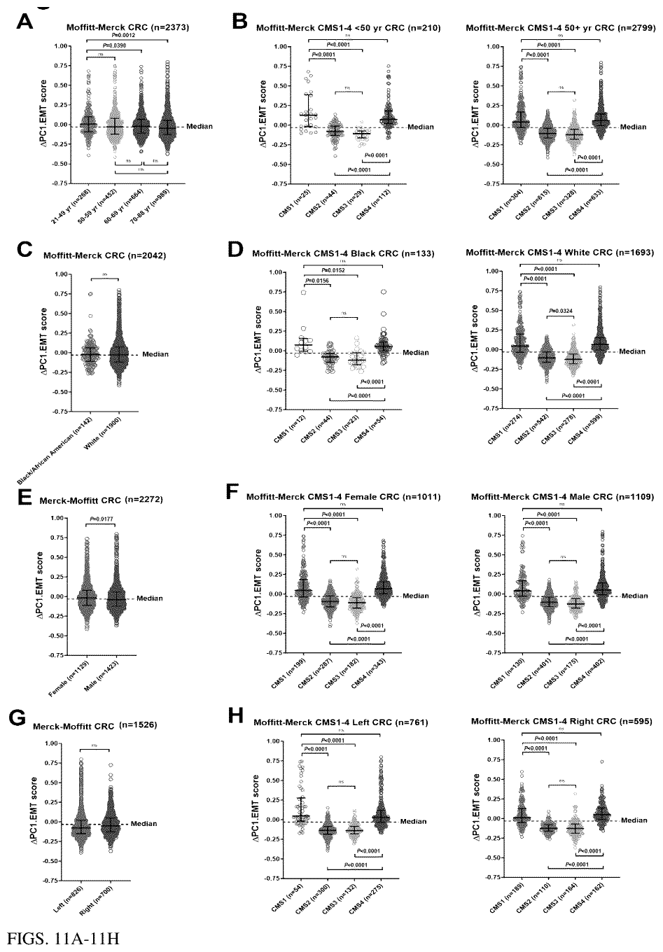

It was previously reported that an epithelial-to-mesenchymal transition (EMT)-based gene expression signature score was highly correlated to the PC1 (the first principal component) signature score in human CRC tumors. A composite signature score (ΔPC1.EMT) was generated by subtracting the EMT score from the PC1 score. As a result, the ΔPC1.EMT score demonstrated a dramatically improved capacity to predict metastasis and clinical outcomes over its parent scores (either PC1 or EMT) and ten other published scores. However, the biology underneath this composite score is still poorly understood. To better understand the biology underpinning this complex ˜500 gene score, the ΔPC1.EMT score was re-analyzed in the context of various baseline characteristics and the CMS classes. Here the Welch's t test was used for score comparison. Higher ΔPC1.EMT scores were significantly (P<0.0001) associated with higher stages (IV>III>II>I) tumors (including both primary and metastatic) and metastatic tumors themselves, and to a lesser extent, with MSI tumors (FIGS. 1A-1C). The Kaplan Meier (KM) survival analysis was also performed by ΔPC1.EMT score quartiles, which confirmed the score to be highly prognostic (FIGS. 1A-1C). Moreover, the ΔPC1.EMT scores were remarkably higher in the CMS1/CMS4 than CMS2/CMS3 classes when compared across all stages, individual stages (I, II, III, IV), primary and metastatic tumors, or MSI and MSS tumors (FIGS. 1D-1F). In addition, among the subgroups of age, sex, race and sidedness, higher scores appeared to associate with younger patients of <50 yr (all stages considered) (FIGS. 11A-11H). Again, strong associations with the CMS1/CMS4 (vs. CMS2/CMS3) subtypes were observed in all the subgroups tested (FIGS. 11A-11H). Taken together, the highly prognostic ΔPC1.EMT score was strongly associated with CMS1 and CMS4 subtypes independent of age, sex, race, stages, MSI, metastasis and sidedness in 2373 CRCs. This strongly suggests that the biology underpinning the ΔPC1.EMT score may be highly related to the distinct cellular features of the CMS1/CMS4 vs. CMS2/CMS3 classes.

### Example 2: While ΔPC1.EMT's Top 10 Positively Correlated (POS) Genes were Predominantly Associated with the CMS1/CMS4 Subtypes that Portended Worse Survival, its Top 10 Negatively Correlated (NEG) Genes were Strongly Related to the CMS2/CMS3 Subtypes that Portended Better Survival

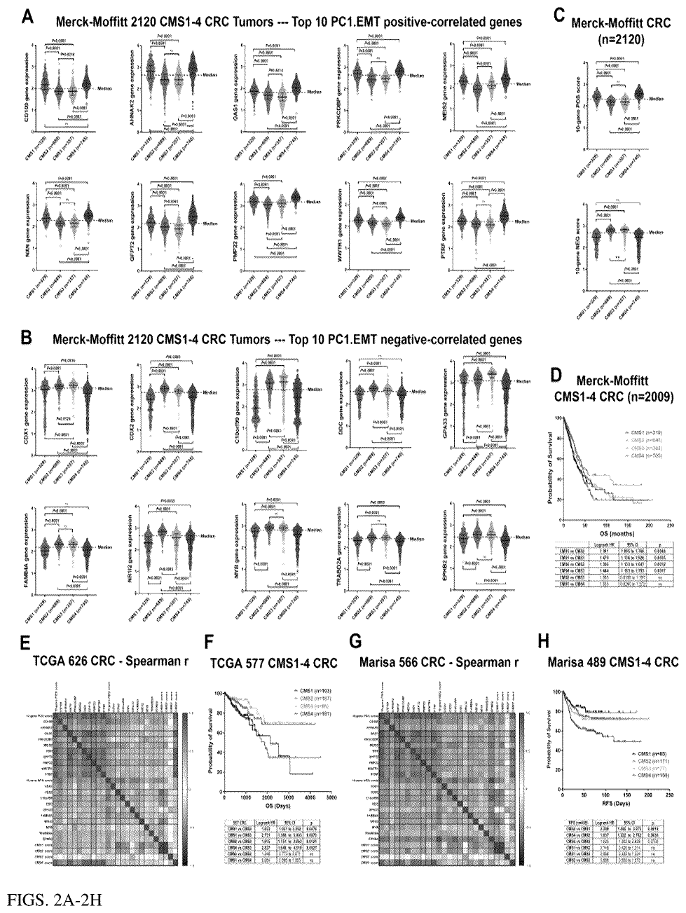

The composite construction of the ˜500-gene ΔPC1.EMT signature score (a combination of 125 PC1-UP genes, 120 PC1-DOWN genes, 150 EMT-UP genes and 162 EMT-DOWN genes), made it difficult to dissect its complex biology. Thus, ΔPC1.EMT's most-correlated genes with known biological functions were further investigated. From an analysis of six independent CRC datasets, it was previously conscripted that the 10 top-ranked, positively-correlated (POS) genes (CD109, AHNAK2, GAS1, PRKCDBP (i.e. CAVIN3), MEIS2, NXN, GFPT2, PMP22, WWTR1, PTRF (i.e. CAVIN1)) (adj P<0.0001) and 10 top-ranked, negatively-correlated (NEG) genes (CDX1, CDX2, C10orf99, DDC, GPA33, FAM84A (i.e. LRATD1), NR1I2, MYB, C2orf89 (i.e.TRABD2A), EPHB2) (adj P<0.0001) Of note, these 20 top genes are mostly related to cancer and metastasis. Remarkably, all top 10 POS genes had significantly higher gene expression in CMS1 and CMS4 tumors (Welch's t test P<0.0001), whereas each of the 10 top NEG genes had significantly higher gene expression in CMS2 and CMS3 tumors (P<0.0001) (FIGS. 2A and 2B). This prompted us to generate a 10-gene POS signature score and a 10-gene NEG signature score (FIG. 2C).

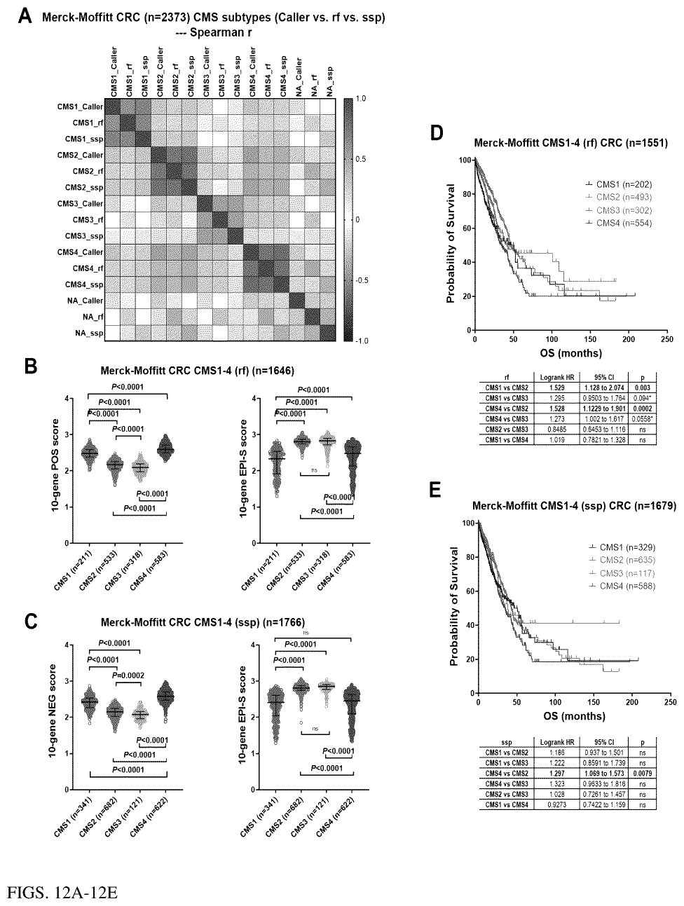

In addition, the CMS1-4 subtypes were generated by the random forest (rf) and single-sample predictor (ssp), the original CMS classifiers reported by Guinney et al., respectively (FIGS. 12A-12E). Notably, the degree of Spearman correlation between the CMScaller and the rf or between the CMScaller and the ssp were the same as between the two original classifiers (rf and ssp) (FIG. 12A), validating the use of the CMScaller in the primary tumors. Similar distinct association of the 10-gene POS signature score vs. the 10-gene NEG signature score with the CMS1-4 subtypes was also obtained by the rf and ssp classifiers (FIGS. 12B and 12C). Moreover, these distinct associations were independently validated in the TCGA (n=626) and Marisa (n=566) CRC datasets (FIGS. 2E and 2G).

Furthermore, the KM analysis among the CMS1-4 classes revealed that both CMS1 and CMS4 classes portended worse OS in the Merck-Moffitt tumors (FIG. 2D (the CMScaller), FIG. 12D (the rf). More distinct OS differences between CMS1/CMS4 vs. CMS2/CMS3 tumors were noted in the TCGA dataset (FIG. 2F) whereas worse RFS was only seen for CMS4 in the Marisa dataset (FIG. 2H). These data suggest that the 10 POS (vs. the 10 NEG) signature genes/score were strongly associated with the CMS1/CMS4 (rather than CMS2/CMS3) subtypes, which portended worse survival.

### Example 3: CIBERSORT Deconvolution Cell Type Analysis Confirmed that the POS Vs. The NEG Signature Genes/Scores were Differentially Associated with Variable Immune/Stromal Cellular Features

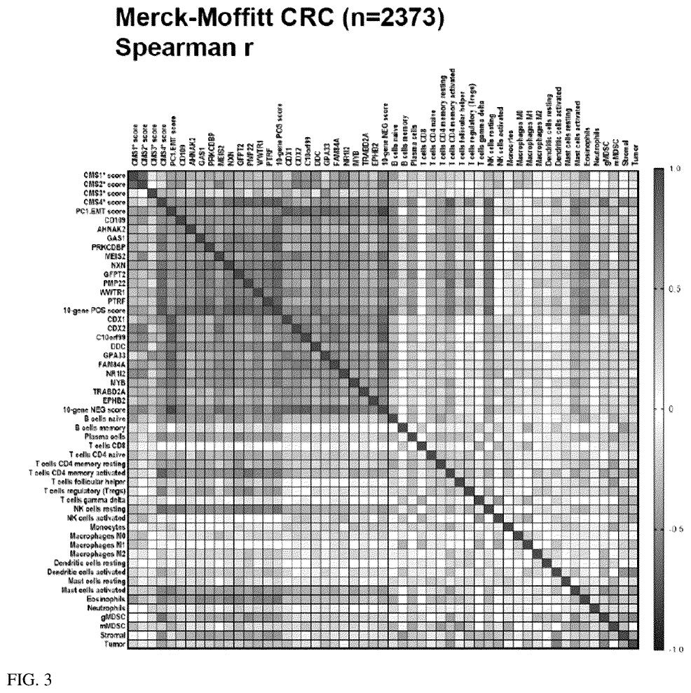

To investigate whether the POS vs. the NEG genes/scores might capture the distinct cellular features associated with the CMS subtypes, CIBERSORT deconvolution analysis was performed in Merck-Moffitt 2373 CRC tumors (FIG. 3). A few key observations were noted:

(1) For the majority of CIBERSORT-defined immune cell populations, similar correlation patterns were seen between CMS1 and CMS4, or between CMS2 and CMS3. While CMS1*/CMS4* scores had negative correlations, the CMS2*/CMS3* scores had positive or no correlations with the cell scores of “B cells naïve”, “Plasma cells”, “T cells CD4 naïve”, “T cells CD4 memory resting”, “T cells CD4 memory activated”, “T cells follicular helper”, “T cells regulatory (Tregs)”, “NK cells resting”, “Mast cells resting”, “Mast cells activated” and “Eosinophils”. In addition, while the CMS2*/CMS3* scores had negative correlations with “Monocytes”, “Macrophages M2”, “Dendritic cells resting”, and “mMDSC”, CMS1*/CMS4* scores had positive correlations with them.

(2) Distinct CMS-associated correlations were also observed. (i) CMS1 was positively correlated with immune “active” cell populations including “B cells memory”, “T cells CD8”, “T cells gamma delta”, “NK cells activated”, “Macrophages M1” and “Dendritic cells activated”. (ii) CMS2 and CMS3 tended to correlate with immune “inactive” cells including “B cells naïve”, “NK cells resting” and “Macrophages M0”. (iii) CMS4 was strongly correlated with “stromal” and “tumor” cells.

(3) The POS genes/score tended to correlate with the CMS1/CMS4-related immune/stromal cell populations. On the other hand, the NEG genes/score tended to correlate with the CMS2/CMS3-related immune inactive/poor cell populations.

(4) The 10-gene POS score was correlated stronger with “CMS4” than “CMS1”, suggesting more preferential association with stromal-related immune-suppressive features.

### Example 4: Analysis of an Independent scRNASEQ Dataset Validated that the 10 POS Genes were Principally TME-Associated Genes, and the 10 NEG Genes were Predominantly EPI-Associated Genes

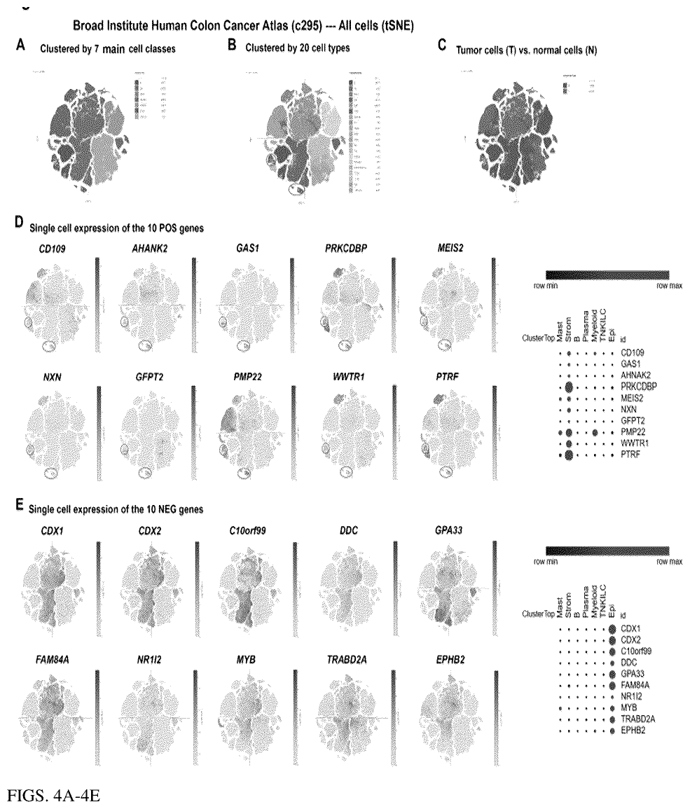

To determine the precise cellular origin of the gene transcripts for the POS/NEG signatures, their expression levels were assessed at single cell resolution using a public colon cancer scRNAseq dataset (Pelka et al. n=62) (FIGS. 4A-4E). Strikingly, all 10 NEG signature genes were consistently and predominantly expressed in the epithelial tumor cells and epithelial normal mucosa, whereas each of the 10 POS signature genes were preferentially expressed in various stromal/immune TME cells (FIGS. 4D and 4E). For example, all of the 10 POS genes were strongly expressed in the fibroblast cells, whereas 9 of the 10 POS genes (except GFPT2) had abundant expression in endothelial cells. Of note, PMP22 showed strong expression in the macrophages. Thus, the 10 POS genes were preferentially expressed by the stromal (and to a lesser extent, immune) TME. In striking contrast, each of the 10 NEG genes was predominantly expressed in epithelial (EPI) tumor cells. The distinct EPI vs. TME expression patterns led us to rename the NEG signature as “EPI^{S}” and the POS signature as “TME^{S}”.

### Example 5: The TME^{S} Vs. The EPI^{S} Genes, and their Respective 10-Gene Signature Scores, Were Distinctly Correlated with BRAF and APC Mutations

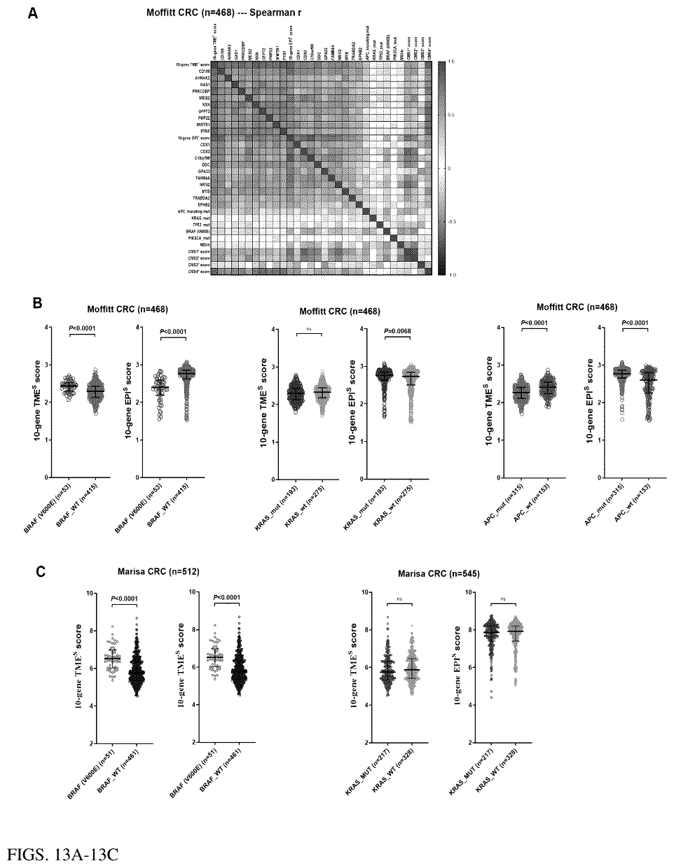

DNA sequencing of 1321 cancer-related genes (including key driver genes such as APC, TP53, KRAS and BRAF) and MSI analysis on a set of 468 clinically characterized, sporadic, colorectal tumors carried out previously is considered. The correlation of key driver mutations were assessed with the TME^{S} vs. the EPI^{S} genes/scores on the 468 tumors (FIG. 13A). The TME^{S} score/genes were positively correlated with BRAF (V600E) and MSI and negatively correlated with APC truncating mutations, whereas the opposite was seen for the EPI^{S} score/genes. Of note, only the BRAF(V600E) and APC truncating mutations were considered as functional mutations for these two driver genes. The distinct correlation with the BRAF vs. APC mutations was clearly illustrated by score comparison analyses against the prevalence of these mutations (FIGS. 13B and 13C). Notably, the CMS correlations were seen for BRAF (V600E) with CMS1* score and for APC mutations with CMS2* score (FIG. 13A).

### Example 6: The TME^{S} and EPI^{S} Genes/Scores were Associated with Distinct Prognostic Contributions

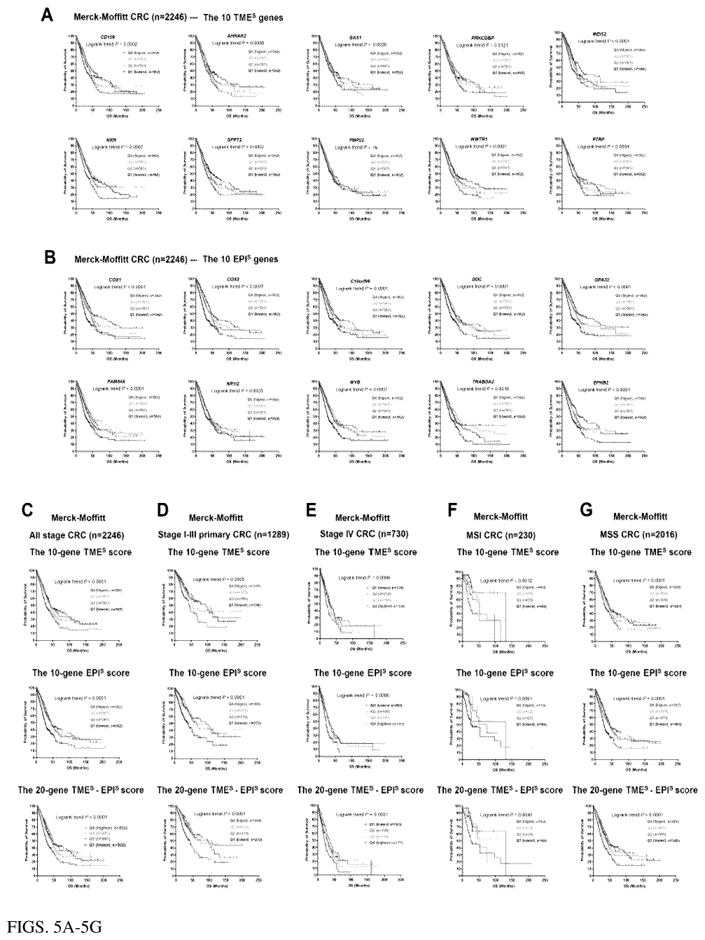

The KM survival analyses is performed to assess if the TME^{S} vs. the EPI^{S} genes/scores might be prognostic in the Merck-Moffitt 2246 tumors that had available OS data. Remarkably, higher expression of 9 of the 10 TME^{S} genes portended worse OS, whereas higher expression of all the 10 EPI^{S} genes were associated with better OS (FIGS. 5A and 5B). Moreover, higher TME^{S} scores and lower EPI^{S} scores were highly prognostic in all stages, Stage I-III primary and MSS tumors, and to a lesser extent, predicted worse OS in MSI and metastatic tumors (FIGS. 5C-5G). These data suggest the TME^{S} vs. the EPI^{S} genes/scores were associated with distinct prognostic contributions. Of note, the 20-gene TME^{S}-EPI^{S} composite score (generated by subtracting the EPI^{S} score from the TME^{S} score) did not further improve prognostic prediction.

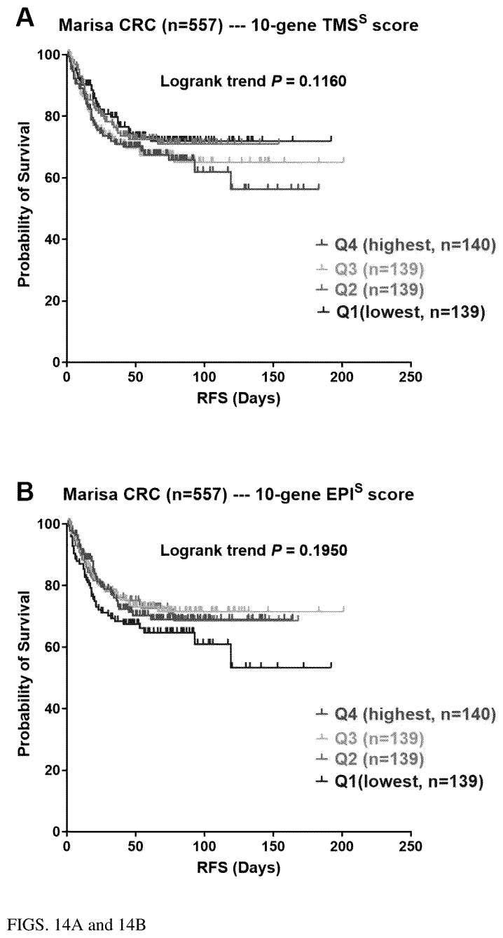

In addition, the KM survival analyses are performed on the 10-gene TME^{S} score and the 10-gene EPI^{S} score in the Marisa CRC tumors that had corresponding RFS data (n=557) (FIGS. 14A and 14B). While the log rank trend p values were not significant, the plots show a trend for higher TME^{S} scores (FIG. 14A, higher quartiles Q4, Q3 vs. lower quartiles Q2, Q1) and lower EPI^{S} scores (FIG. 14B, the lowest quartile Q1 vs. other quartiles Q2-4) to be associated with worse RFS.

### Example 7: Multivariable Cox Regression Analyses Against the 29 Known TME-Signatures Revealed that while the TME^{S} and the EPI^{S} were Independent Prognostic Signatures, the TME^{S} Score was Strikingly Impacted by the “Cancer-Associated Fibroblasts” Signature

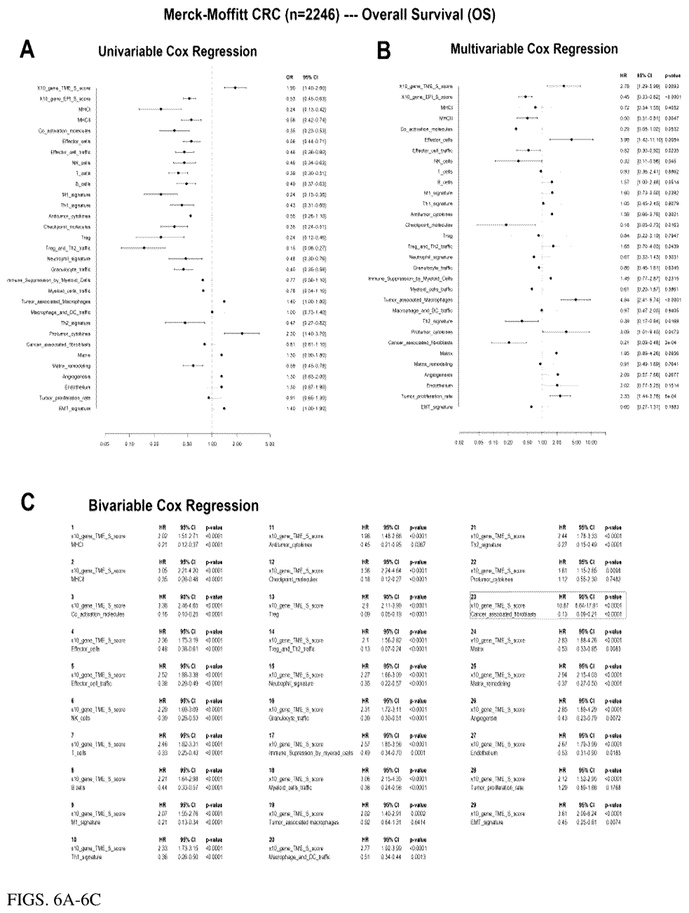

Bagaev et al. recently established a list of 29 TME gene expression signatures (Fges) representing the major functional components and immune, stromal and other cellular populations of a tumor for a pan-cancer TME subtyping analysis. To determine whether the 10-gene TME^{S} and the 10-gene EPI^{S} signatures were independent prognostic signatures, a multivariable Cox regression analysis was performed against the 29 TME-Fges in the Merck-Moffitt CRC dataset (FIG. 6B). A univariable Cox regression analysis was also performed of these signatures as a reference (FIG. 6A). Both the TME^{S} and the EPI^{S} were shown to be independent prognostic signatures when analyzed along with the 29 TME-Fges (FIG. 6B). Moreover, bivariable Cox analyses of the TME^{S} signature confirmed the TME^{S} signature as an independent prognostic signature when competing against each of the 29 Fges, respectively (FIG. 6C). Remarkably, the TME^{S} signature was strikingly impacted by the “Cancer-associated fibroblasts” (CAF) signature (HR: 10.87 [6.64-17.81] for TME^{S} vs. 0.13 [0.09-0.21] for CAF, both P<0.0001) when compared to their univariable Cox OR (1.90 [1.40-2.60] for TME^{S} and 0.81 [0.61-1.10] for CAF). This is supported by the single cell gene expression data showing that all the 10 genes were predominantly expressed in stromal/fibroblast cell types (FIG. 4D). In addition, to a lesser degree, the TME^{S} signature was also considerably impacted by a number of other Fges (e.g. HR: 3.38 [2.46-4.65] for TME^{S} vs. 0.16 [0.10-0.25] for “Co-activation molecules”, both P<0.0001; HR: 3.36 [2.44-4.64] for TME^{S} vs. 0.18 [0.12-0.27] for “Checkpoint molecules”, both P<0.0001; HR: 3.61 [2.09-6.24], P<0.0001 for TME^{S} vs. 0.45 [0.25-0.81] for “EMT-signature”, P=0.0074). These data suggest that the 10-gene TME^{S} signature could capture various stromal and immune TME components. These data are in agreement with the strong association of the TME^{S} signature with both immune CMS1 and stromal CMS4 subtypes that portended worse survival (FIG. 2A).

### Example 8: The TME^{S} and the EPI^{S} Scores were Distinctly Correlated with the iCMS3 Signature Score

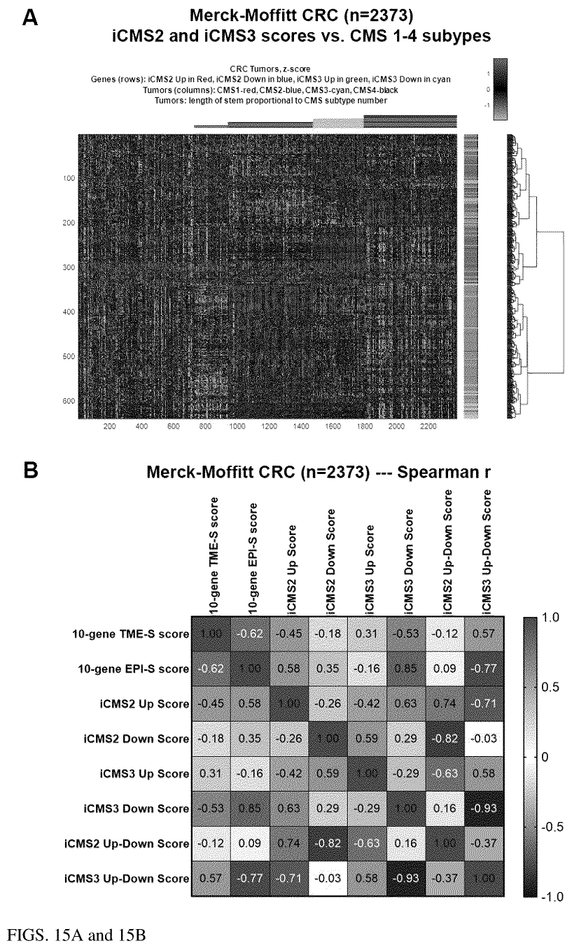

Joanito et al. recently identified two intrinsic (epithelial) subtypes (iCMS2 and iCMS3) that refined CMS classification. The iCMS3 subtype, that comprised MSI tumors and also one-third of MSS tumors, portended worse prognosis (vs. the iCMS2 subtype). Notably, the iCMS3 associated with the CMS4 had the worst prognosis. A heatmap correlation analysis was performed on the iCMS2/iCMS3 signatures vs. the CMS subtypes in the Merck-Moffitt tumors (n=2373) (FIGS. 15A and 15B). The iCMS2 up genes were primarily correlated with the CMS2 subtype, supporting the most prominent feature of the iCMS2 (FIG. 15A). However, the iCMS2 down genes, the iCMS3 up and down genes had less clear, “mixed” correlations with CMS1/CMS3/CMS4 (FIG. 15A). Further signature score correlation analysis shows that the 10-gene TMS^{S} score, and the 10-gene EPI^{S} score had no correlation with the iCMS2 (up—down) signature score (FIG. 15B). By contrast, the TMS^{S} score, and the EPI^{S} score were distinctly correlated with the iCMS3 (up—down) signature score (Spearman r=0.57 for the TMS^{S} vs. r=−0.77 for the EPI^{S}) (FIG. 15B). Since the iCMS3 was associated with worse prognosis, this supports the distinct association of the TMS^{S}(worse) vs. the EPI^{S}(better) with CRC prognosis.

### Example 9: The TME^{S} and the EPI^{S} Scores were Distinctly Associated with the PDS1-3 Subtypes

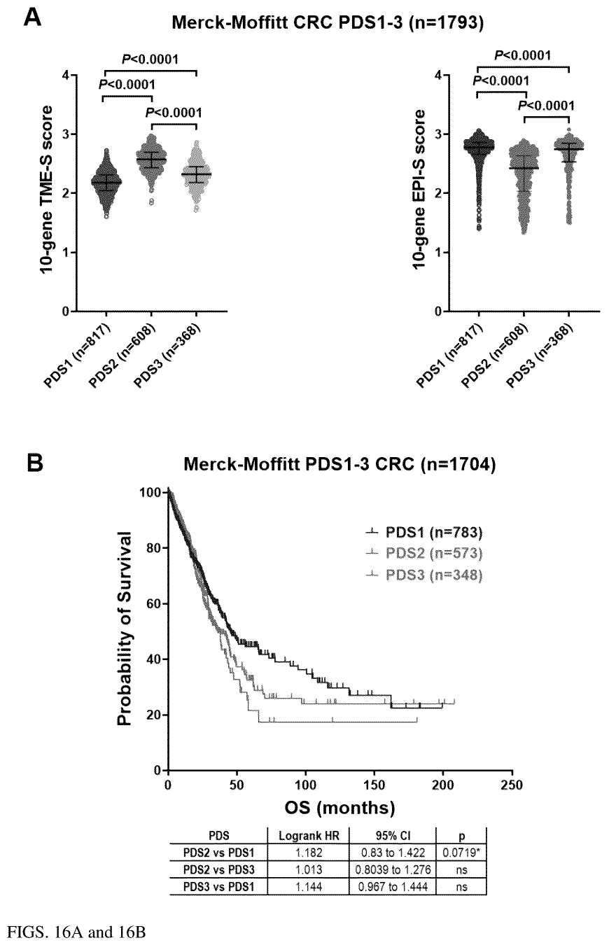

Malla et al. recently performed pathway level subtyping of CRC and defined three pathway-derived subtypes (PDS1-3): PDS1-canonical/LGR5⁺ stem-rich and highly proliferative, with good prognosis; PDS2-regenerative/ANXA1⁺ stem-rich, with elevated stromal and immune TME lineages; PDS3—a slow-cycling subset of CMS2 tumors with reduced stem populations and increased differentiated lineages and with the worst prognosis in locally advance disease. The Merck-Moffitt tumors (n=2373) were classified into the PDS1 (n=817), the PDS2 (n=608) and the PDS3 (n=368) as well as the mixed subtype (n=580). The Welch's t test was performed to compare the 10-gene TMS^{S} score, and the 10-gene EPI^{S} score among the PDS1-3 subtypes, respectively (FIGS. 16A and 16B). Significant score differences were seen for both the TMS^{S} and the EPI^{S} but with opposite directionalities (PDS2>PDS3>PDS1, all P<0.0001 for the TMS^{S} vs. PDS2<PDS3<PDS1, all P<0.0001 for the EPI^{S}). Notably, the highest TMS^{S} score vs. the lowest EPI^{S} score in the PDS2 “TME” subtype agrees with the distinct association of the two scores with the TME. In addition, the lowest TMS^{S} score vs. the highest EPI^{S} score in the PDS1 (good prognosis) subtype is also consistent with the distinct association of the two scores with CRC prognosis (see FIGS. 5C-5G). Of note, the KM survival analysis of the PDS1-3 subtypes in the Merck-Moffitt tumors shows that while not significant, the PDS1 tended to have better OS (FIG. 16B).

### Example 10: The TME^{S} Score was Strongly Correlated with EMT, SRC Activation and MEK Inhibitor Resistance

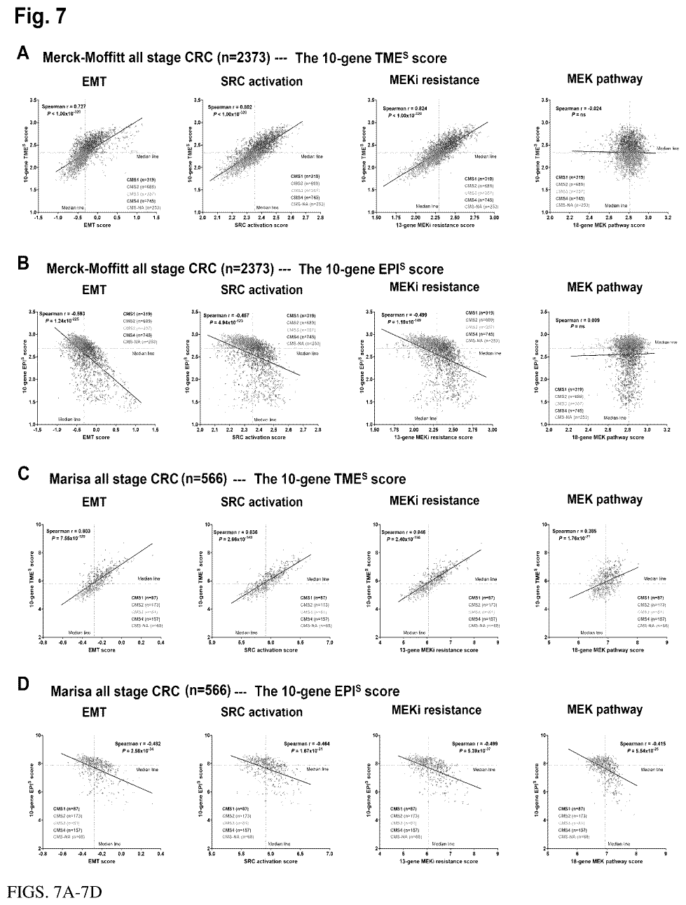

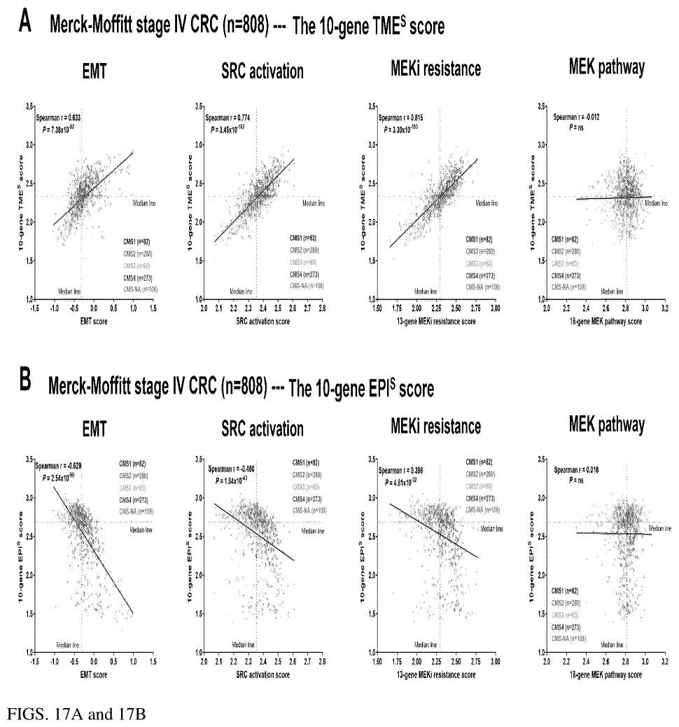

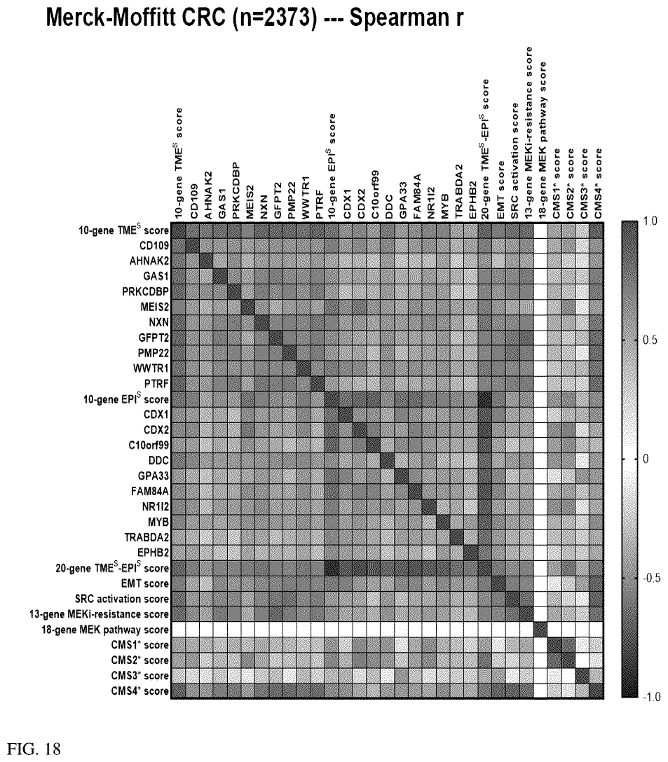

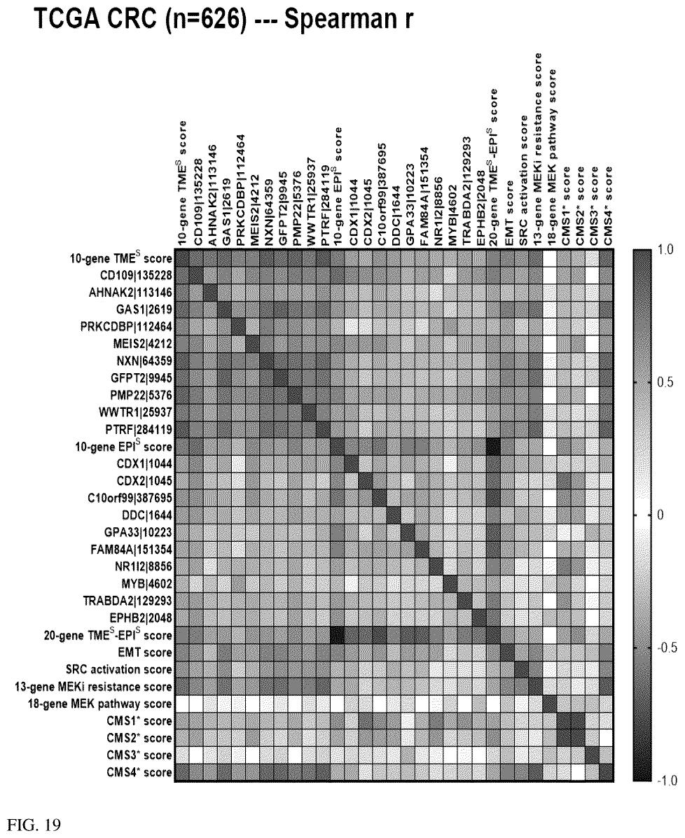

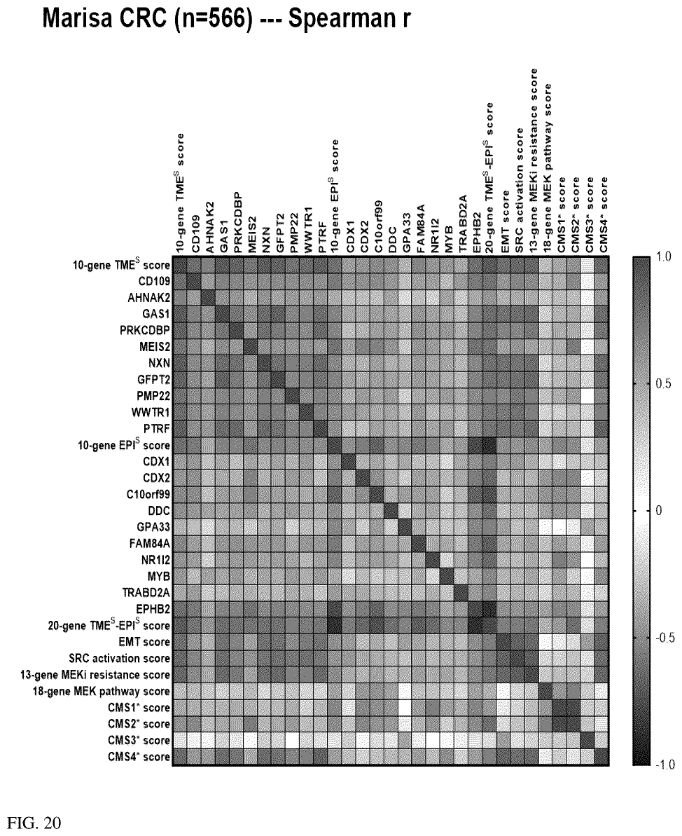

Over half of CRC tumors are driven by oncogenic RAS/RAF mutations, suggesting that CRC is hard-wired to RAS/RAF/MEK signaling. KRAS/BRAF-mutated CRC has been targeted but therapeutic inhibitors are largely ineffective, likely due to complex resistance mechanisms that are not well understood. It was reported previously that the SRC activation signature score was strongly correlated with the 13-gene MEKi-resistance signature score (that predicts drug resistance caused by “bypass” proliferation/survival signaling pathways). It is now identified that the TME^{S} signature score was highly correlated with the EMT, SRC activation and 13-gene MEKi-resistance signature scores in Merck-Moffitt 2373 CRC tumors (Spearman r=0.727, 0.802, 0.824, respectively, all P<10⁻³²⁰, see FIG. 7A). By contrast, the EPI^{S} signature score was, to a lesser statistical degree, negatively correlated with these scores (FIG. 7B). Notably, CMS4 (and to a lesser extent, CMS1) tumors were strongly associated with higher scores of EMT, SRC activation and MEKi-resistance, whereas CMS2 and CMS3 tumors were highly associated with their lower scores (FIGS. 7A and 7B). Moreover, these findings were validated in the Merck-Moffitt Stage IV tumors (FIGS. 17A and 17B) and the Marisa CRC dataset (n=566) (FIGS. 7C and 7D). Furthermore, the same distinct correlation patterns were also seen for all the TME^{S} vs. the EPI^{S} genes in multiple datasets (FIGS. 18, 19 and 20), suggesting a contributive role of the individual genes.

### Example 11: The EPI^{S} Score was Associated with Longer Progression Free Survival in Cetuximab-Treated Metastatic CRC Patients Derived from Two Independent Clinical Trials

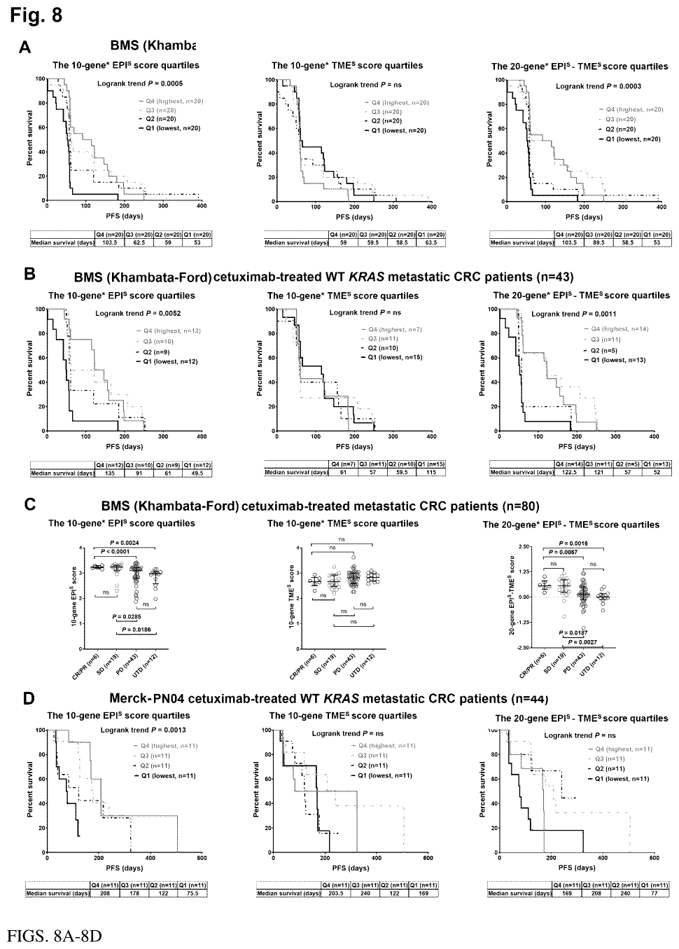

The potential of the EPI^{S} score vs. the TME^{S} score was examined to predict outcomes in metastatic CRC patients treated with cetuximab in two independent clinical trial datasets (BMS (Khambata-Ford), Merck (PN04)). Higher EPI^{S} scores were highly associated with longer progression free survival (PFS) in the BMS dataset with all patients regardless of KRAS mutation status) (n=80, Logrank trend P=0.0005, median survival (days): 103.5 (Q4, highest) vs. 53 (Q1, lowest)) and in the subset of WT KRAS patients (n=43, P=0.0062, median survival (days): 135.0 (Q4) vs. 49.5 (Q1)) (FIGS. 8A and 8B). The TME^{S} score, by itself, was not predictive of CTX sensitivity, and it did not add any predictive value to the EPI^{S} score when combined with it (20-gene EPI^{S}/TME^{S} score) (FIGS. 8A and 8B). Of note, higher EPI^{S} scores were significantly associated with patients with complete/partial responses (CR/PR) and stable disease (SD) (FIG. 8C). The same predictive value was also demonstrated in the Merck-PN04 dataset with WT KRAS patients (n=44, P=0.0013, median survival (days): 208.0 (Q4) vs. 75.5 (Q1)) (FIG. 8D).

### Example 12: Establishing the Association of EPI^{S} and TME^{S} Scores with OS, EGFRi-OS And EGFRi-TOT in a Large Real World Clinic-Genomics Dataset

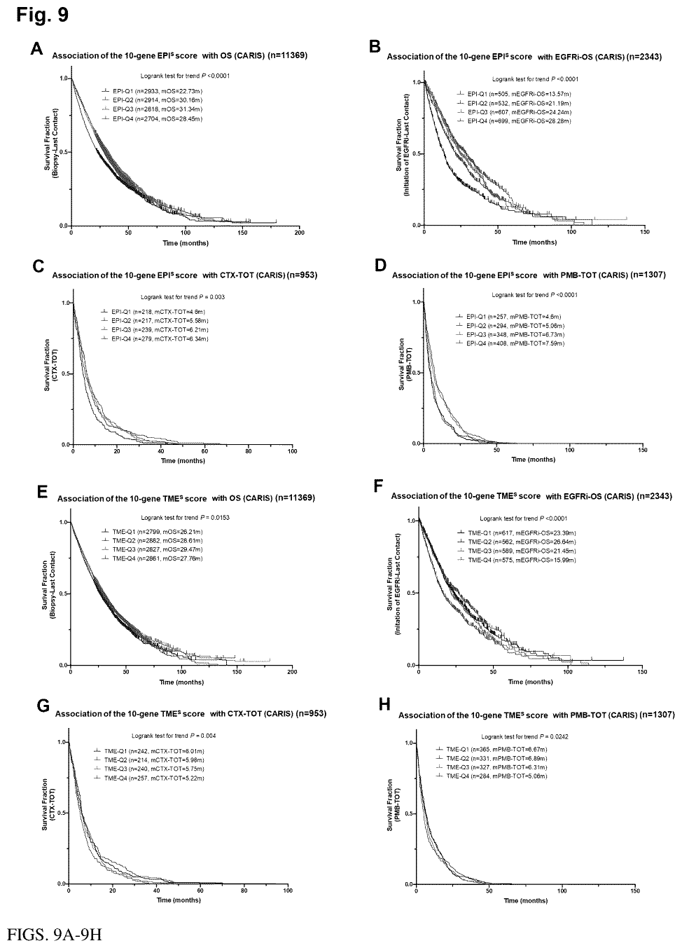

Finally, using a large real-world dataset, the association of the EPI^{S} score and TME^{S} score with clinical outcomes was investigated. The KM analysis showed that the EPI^{S} score modestly but very significantly (Logrank trend P<0.0001) predicted better OS whereas the TME^{S} score was barely prognostic (P=0.0153) on all CRC patients excluding those treated with EGFRi (n=11369, FIGS. 9A and 9E). The association between the EPI^{S} and TME^{S} scores with survival from the initiation of EGFRi (OS) and time on treatment (TOT) was investigated as a surrogate for PFS in patients treated with cetuximab (CTX) or panitumumab (PMB). These treatments were delivered in all lines of therapy. It was found that higher EPI^{S} scores were associated with increased OS (n=2343, P<0.0001) and TOT with EGFRi therapy (CTX, n=953, P=0.003; PMB, n=1307, P<0.0001) (FIGS. 9B-9D). On the other hand, higher TME^{S} scores were significantly, but to a lesser extent, associated with decreased OS (P<0.0001), CTX-TOT (P=0.004) and PMB-TOT (P=0.0242) (FIGS. 9F-9H).

### DISCUSSION

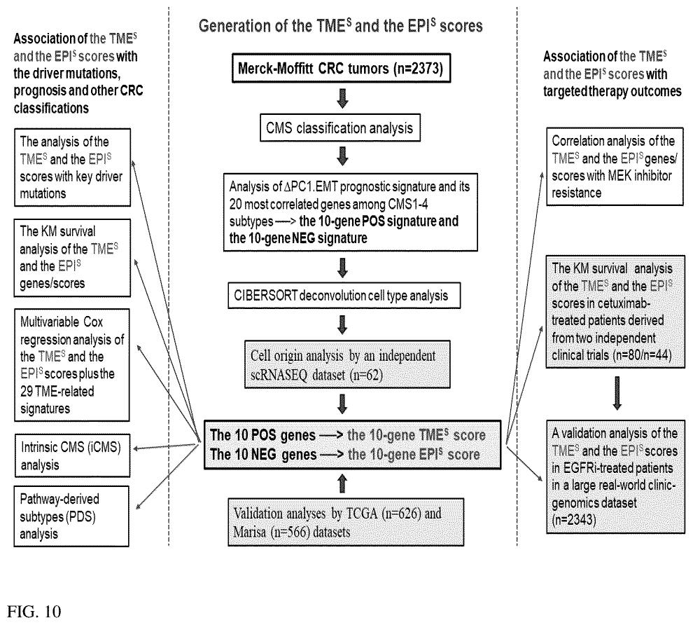

Cancer progression and therapeutic response depend on both tumor epithelium (EPI) and their surrounding immune/stromal TME. However, the differential contribution of cell type and relative geography of the EPI vs. the TME to CRC prognosis and therapeutic outcomes is not yet clearly defined. An analysis of 2373 human colorectal cancers validated by multiple independent CRC datasets (FIG. 10 for study flowchart), leading to development of a companion pair of distinct 10-gene signatures (TME^{S} vs. EPI^{S}) scores capable of distinguishing the cellular origin and quantitative contribution of epithelial tumor vs. the TME to both prognostic and therapeutic outcomes is reported.

It is deciphered that the biology underpinning the complex ΔPC1.EMT score. The ΔPC1.EMT score and its most correlated genes (10 POS genes vs. 10 NEG genes) had distinct correlations with the TME-rich CMS1 and CMS4 (associated with worse survival) vs. the TME-poor (epithelial) CMS2 and CMS3 subtypes (associated with better survival) (FIGS. 1A-1C and 2D). Notably, higher ΔPC1.EMT scores appeared to be associated with younger patients of <50 yr (all stages considered) (FIG. 11A). It is noteworthy that the CRC incidence was increasing in young patients, which was higher for African Americans as reported by Ashktorab et al. The analysis also shows that the “Black or African American” patients had lower CMS1 tumors (4%, P<0.05) (Table 1) but more CRC classification analyses are needed for African Americans. The differential association of the 10 POS genes/score vs. the 10 NEG genes/score with variable immune/stromal TME cellular features was demonstrated by CIBERSORT deconvolution analysis (FIG. 3). Next, scRNASEQ analyses (FIG. 4D) both confirmed that the 10 POS genes, that were strongly correlated with the CMS1/CMS4 subtypes, represented TME associated genes (TME^{S}). In striking contrast, the 10 NEG genes, that were strongly correlated with the CMS2/CMS3 subtypes, represented EPI associated genes (EPI^{S}).

The data revealed that the TME^{S} genes/score predicted worse OS (FIG. 5A). This is supported by the observation that all the 10 TME^{S} genes were principally expressed in the tumor stroma, primarily in the tumor fibroblasts (FIGS. 4B and 4D). The tumor stroma can account for up to >50% of tumor mass of CRC and its extent is predictive of worse survival. Cancer associated fibroblasts, the dominant cellular components of tumor stroma, were reported to promote tumor cell invasion and metastasis. The multivariable Cox regression analyses against the 29 TME gene expression signatures in the Merck-Moffitt tumors show the HR of the TME^{S} score was the most strikingly impacted by the “Cancer associated fibroblasts” signature (FIG. 6C). The TME induces EMT in various types of tumor cells. EMT is aberrantly activated in cancer as a major mechanism promoting invasion and metastasis and is also known to contribute to drug resistance. It was found that the 10 TME^{S} genes and their composite score were strongly correlated with the EMT score and the “mesenchymal” CMS4 subtype that portended worse survival (FIGS. 2A, 2E, 2G, 7A, 7C 17A, 17B, 18, 19, and 20).

Intriguingly, the 10 TME^{S} individual genes and their signature score were also strongly associated with the CMS1 (MSI, immune) subtype (FIGS. 2A, 2E, 2G, 3 and 11A-11H). The multivariable Cox analyses also show that the HR of the TME^{S} score was also considerably impacted by various “immune” signatures including the “Co-activation molecules” and “Checkpoint molecules” signatures that were associated with better OS (FIGS. 6A-6C). These data suggest that the 10-gene TME^{S} signature could capture various stromal and immune TME components. The “MSI, immune” CMS1 subtype was associated with response to checkpoint inhibitor therapy (e.g. pembrolizumab). However, despite its positive association with immunotherapy, the CMS1 subtype portended worse prognosis (OS) in the Merck-Moffitt CRC tumors (n=2009) (FIG. 2D). The association of CMS1 with worse prognosis was also recently reported by Chowdhury et al. when analyzed in all CRC (n=24,939), primary or local tumors (n=14,153) and distant metastatic tumors (n=10,776). Why the CMS1 tumors were associated with worse prognosis is largely not clear. The CMS1 tumors are less metastatic (Table 1) and confer the best prognosis in early-stage CRCs but portend very poor outcomes at advanced stages. Moreover, BRAF(V600) mutation was the most frequently present in CMS1. BRAF(V600E) was reported as a strong negative prognostic marker of CRC but the biology is poorly understood. Notably, it was found that the TME^{S} genes and score were positively correlated with BRAF (V600) (FIGS. 13A-13C), suggesting a potential biological role of the TME in BRAF (V600)-mediated poor prognosis. Moreover, the TME^{S} score was strongly related to the stromal/fibroblast TME (FIGS. 4D and 6C) and the fibrotic subtypes were reported to associate with the non-responders in immunotherapy, suggesting that the TME^{S} score may not have association with immunotherapy response. On the other hand, the data revealed that the TME^{S} genes/score were strongly associated with SRC activation and MEK inhibitor resistance and (FIGS. 7A, 7B, 17A, 17B, 18, 19, and 20). Of note, the SRC oncogene is a non-receptor tyrosine kinase that mediates cell proliferation, survival, invasion and motility and is regulated by the TME.

By contrast, the 10-gene EPI^{S} genes and their composite score predicted better OS (FIG. 5B). The EPI^{S} score was associated with longer PFS in cetuximab-treated metastatic CRC patients in two clinical trial datasets (FIGS. 8A-8D). Furthermore, it was independently validated that the EPI^{S} score is mildly prognostic (better OS) but highly predictive of EGFRi outcomes (better OS and longer TOT) using a large real world CarisLS dataset (FIGS. 9A-9D). Notably, the TME^{S} score was also prognostic and predictive (worse outcomes) but to a lesser degree (FIGS. 9E-9H). In addition, the EPI^{S} score was significantly correlated with APC-truncating mutations (FIGS. 13A-13C) that may predict cetuximab sensitivity. These data suggest clinical potential for this 10-gene EPI^{S} signature as a predictive biomarker to improve EGFRi outcome.

In addition to the CMS classification, other new CRC classifications have been recently developed as well. For example, Joanito et al. identified two intrinsic (epithelial) subtypes (iCMS2 and iCMS3), whereas Malla et al. defined three pathway-derived subtypes (PDS1-3). The additional analyses in the Merck-Moffitt CRC tumors revealed that 10-gene TME^{S} score vs. the 10-gene EPI^{S} score were distinctly associated with the iCMS3 signature and with three PDS subtypes (FIGS. 15A, 15B, 16A and 16B). These data also support the association of the 10-gene TME^{S} vs. the 10-gene EPI^{S} with distinct prognostic contributions.

Remarkably, nine of the 10 TME^{S} genes and all the 10 EPI^{S} genes were prognostic (FIG. 5A), suggesting individual functional importance. The biological roles of many of these genes have been reported. For example, CD109 (a TME^{S} gene) was identified as a metastasis-associated protein marker. Reduced expression of EPHB2 (an EPI^{S} gene) was associated with metastasis and its overexpression induced EMT. How the biological functions of the TME^{S} vs. EPI^{S} genes are related to their distinct contributions to CRC outcomes remains to be investigated.

### CONCLUSIONS

This study has generated a pair of new, distinct 10-gene signature scores (the TME^{S} vs. the EPI^{S}) that identify differential cellular contributions of the TME and the tumor epithelium to therapeutic outcomes. While the TME^{S} score was strongly correlated with MEKi resistance, the EPI^{S} score was highly predictive of EGFRi outcomes as demonstrated in clinical trial and large real-world datasets. With targeted approaches emerging to address both the TME and the tumor EPI, the distinct 10-gene signature scores (the TME^{S} vs. the EPI^{S}) may have a novel biomarker role to permit optimization of CRC therapy by identifying resistant vs. sensitive subpopulations.

### Methods

A flowchart of this study is shown in FIG. 10, which illustrates generation and analysis of the 10-gene TME^{S} versus the 10-gene EPI^{S} signature scores in the CRC dataset and various other CRC datasets.

CRC Dataset (n=2373)

The cohort of 2373 colorectal adenocarcinoma tumors, including 1571 primary lesions and 802 metastatic lesions (Table 1), were obtained from adult patients treated through the Total Cancer Care initiative, which was created by the H. Lee Moffitt Cancer Center (Tampa, FL). All tumor tissues were collected by microdissection and snap freezing in liquid nitrogen within 15-20 min of extirpation. The Affymetrix gene expression data were obtained as described by the Merck group as a part of the large reference datasets representing >25 different cancers to develop consensus signatures for pembrolizumab response. This study used only retrospective and de-identified clinical data for various bioinformatic analyses. A subset of 468 tumors was previously analyzed with global gene expression data, MSI status, and targeted gene sequencing of 1321 cancer-related genes including key driver genes (BRAF, KRAS, APC and TP53). Correlation analyses of the TME^{S} score vs. the EPI^{S} signature score was performed with these key driver genes in the 468 tumors.

CARIS LS Real-World Clinico-Genomics Dataset with Longitudinal Outcomes

Whole Transcriptome Sequencing (WTS): Formalin fixed paraffin embedded (FFPE) CRC specimens (from 11,369 non-EGFRi and 2,343 EGFRi treated patients) underwent pathology review to measure percent tumor content and tumor size; a minimum of 10% of tumor content in the area for microdissection was required to enable enrichment and extraction of tumor-specific RNA. The Qiagen RNA FFPE tissue extraction kit was used for RNA extraction, and the RNA quality and quantity were determined using the Agilent TapeStation. Biotinylated RNA baits were hybridized to the synthesized and purified cDNA targets, and the bait-target complexes were amplified in a post-capture PCR reaction. The Illumina NovaSeq 6500 was used to sequence the whole transcriptome from tumor samples to an average of 60M reads. Raw data were demultiplexed by Illumina Dragen BioIT accelerator, trimmed, counted, removed of PCR-duplicates, and aligned to human reference genome hg19 by STAR aligner. TPM (Transcripts Per Million Molecules) were generated using the Salmon expression pipeline. The TME^{S}/EPI^{S} genes were log transformed and z-scored. The TME^{S}/EPI^{S} signature scores were calculated as the average z-scores for all the TME^{S}/EPI^{S} genes for a given specimen.

Clinical Outcomes: Real-world overall survival (OS) information was obtained from insurance claims data using Kaplan-Meier estimates. OS/EGFRi-OS was calculated from the time of tissue biopsy/EGFRi to last contact while time on EGFRi treatment (TOT) was calculated from the initiation of Cetuximab/Panitumumab to its termination (Cetuximab-TOT/Panitumumab-TOT). P values were calculated using the log-rank test for trend.

Pelka et al. scRNASEQ CRC Dataset (n=62)

Recently, Pelka et al. used scRNASEQ to transcriptionally profile 371,223 cells from colorectal tumors and adjacent normal tissues of 62 CRC patients. The public available scRNASEQ dataset on Human Colon Cancer Atlas (c295)-Single Cell Portal from Broad Institute was analyzed and visualized to assess the single cell expression of the two distinct 10-gene signatures in epithelial tumor cells vs. immune/stromal cells.

Marisa et al. CRC Dataset (n=566) and TCGA CRC Dataset (n=626)

These two public CRC datasets were accessed as described for the previous validation analyses. Here 19 normal samples were removed from the Marisa dataset (n=585), whereas 51 normal samples were removed from the TCGA dataset (n=677). The Marisa (n=566) and TCGA (n=626) CRC datasets were used to independently validate the distinct gene expression/score/CMS correlations in relation to CRC outcomes and resistance to targeted therapy. In addition, the correlation analyses of the TME^{S} score vs. the EPI^{S} signature score were performed with BRAF(V600E) and KRAS mutations reported in the Marisa et al. CRC dataset.

Merck (PN04, n=44) and BMS (Khambata-Ford, n=80) (Cetuximab) Clinical Trial Datasets

The PN04 dataset had 44 WT KRAS CRC samples selected from the control arm (cetuximab+irinotecan) of a Merck prospective clinical trial (MK0646), whereas the public available Khambata-Ford dataset was from a BMS trial including 80 cetuximab-treated CRC patient samples among which 43 had WT KRAS. The Affymetrix data was used to examine the ability of the 10-gene EPI^{S}(vs. 10-gene TME^{S}) signature score in predicting progression free survival (PFS) in cetuximab-treated metastatic patients.

### Gene Expression Signature Scores

The gene expression signature scores were calculated for each tumor as previously described. Briefly, a score was computed for each of the signatures as the arithmetic mean of all probe sets corresponding to gene symbols present in the corresponding gene signature. Lists of the signature genes used here include previously described ΔPC1.EMT and EMT scores, SRC activation, 13-gene MEKi resistance and 18-gene MEK pathway activation. In addition, the gene signature scores derived from the iCMS2 and iCMS3 signatures were also calculated as reported by Joanito et al. and the 29 TME-related functional gene expression signatures (Fges) reported by Bagaev et al., respectively.

### Statistical Methods

CMS classification: the Merck-Moffitt (n=2373), TCGA (n=626) and Marisa (n=566) CRC tumors were classified by the CMScaller reported by Eide et al. as previously described. The CMS1*, CMS2*, CMS3* and CMS4* scores were generated for each of these CRC tumors as previously described. The CMS1-4* scores were designated to measure the propensity of a tumor to fall into CMS1, CMS2, CMS2 and CMS4 classes, respectively. Moreover, the Merck-Moffitt CRC tumors were also classified by the random forest (rf) and single-sample predictor (ssp), the original CMS classifiers reported by Guinney et al., respectively. Of note, the CMScaller is a cancer cell-intrinsic CMS classifier that could recapitulate the CMS subtypes in both in vitro and in vivo models and also performed well in primary tumors. The analysis in the Merck-Moffitt tumors also showed that the CMS subtypes were well correlated among the three CMS classifiers (CMScaller, the rf and the ssp) (as shown later). Since the CMScaller could classify significantly more tumors into the CMS1-4 subtypes (n=2120) than the rf (n=1646) and the ssp (n=1766), the CMS subtypes generated by the CMScaller were primarily presented in this study.

PDS classification: the Merck-Moffitt CRC tumors (n=2373) were also classified by the pathway-derived subtypes (PDS1-3) as reported by Malla et al.

CIBERSORT is a method for enumeration of cell subsets to characterize cell composition of complex tissues from their gene expression profiles. A CIBERSORT deconvolution analysis of the global expression data was performed on the Merck-Moffitt 2373 CRC tumors similarly as reported.

Statistical analyses, including Kaplan Meier (KM) survival, Spearman correlation, heatmap, Welch's t Test, Cramer's V Test, and CMS and PDS classifications were performed using GraphPad Prism 10.0.2, SAS 9.4 and R version 4.3. The statistical tests with an α=0.05 were chosen as the significance level. In addition, for the Welch t test in comparison among multiple subgroups, adjusted p values after adjustments for multi-comparisons by Holm-Bonferroni method were indicated. All tests were two-sided unless indicated otherwise.

Those skilled in the art will appreciate that numerous changes and modifications can be made to the preferred examples of the invention and that such changes and modifications can be made without departing from the spirit of the invention. It is, therefore, intended that the appended claims cover all such equivalent variations as fall within the true spirit and scope of the invention.

Throughout this application, various publications are referenced. The disclosures of these publications in their entirety are hereby incorporated by reference into this application in order to more fully describe the state of the art to which this pertains. The references disclosed are also individually and specifically incorporated by reference herein for the material contained in them that is discussed in the sentence in which the reference is relied upon.

### REFERENCES

Ashktorab H, Brim H. Colorectal cancer subtyping. Nat Rev Cancer. 2022; 22(2):68-9.
Ashktorab H, Vilmenay K, Brim H, Laiyemo A O, Kibreab A, Nouraie M. Colorectal Cancer in Young African Americans: Is It Time to Revisit Guidelines and Prevention?Dig Dis Sci. 2016; 61(10):3026-30.
Ayers M, Nebozhyn M, Cristescu R, McClanahan T K, Perini R, Rubin E, et al. Molecular Profiling of Cohorts of Tumor Samples to Guide Clinical Development of Pembrolizumab as Monotherapy. Clin Cancer Res. 2019; 25(5):1564-73.
Bagaev A, Kotlov N, Nomie K, Svekolkin V, Gafurov A, Isaeva 0, et al. Conserved pan-cancer microenvironment subtypes predict response to immunotherapy. Cancer Cell. 2021; 39(6):845-65 e7.
Bhowmick N A, Neilson E G, Moses H L. Stromal fibroblasts in cancer initiation and progression. Nature. 2004; 432(7015):332-7.
Bissell M J, Radisky D. Putting tumours in context. Nat Rev Cancer. 2001; 1(1):46-54.
Broecker F, Hardt C, Herwig R, Timmermann B, Kerick M, Wunderlich A, et al. Transcriptional signature induced by a metastasis-promoting c-Src mutant in a human breast cell line. FEBS J. 2016; 283(9):1669-88.
Bruni D, Angell H K, Galon J. The immune contexture and Immunoscore in cancer prognosis and therapeutic efficacy. Nat Rev Cancer. 2020; 20(11):662-80.
Calon A, Lonardo E, Berenguer-Llergo A, Espinet E, Hernando-Momblona X, Iglesias M, et al. Stromal gene expression defines poor-prognosis subtypes in colorectal cancer. Nat Genet. 2015; 47(4):320-9.
Cancer Genome Atlas N. Comprehensive molecular characterization of human colon and rectal cancer. Nature. 2012; 487(7407):330-7.
Caner A, Asik E, Ozpolat B. SRC Signaling in Cancer and Tumor Microenvironment. Adv Exp Med Biol. 2021; 1270:57-71.
Cascio S, Chandler C, Zhang L, Sinno S, Gao B, Onkar S, et al. Cancer-associated MSC drive tumor immune exclusion and resistance to immunotherapy, which can be overcome by Hedgehog inhibition. Sci Adv. 2021; 7(46):eabi5790.
Caunt C J, Sale M J, Smith P D, Cook S J. MEK1 and MEK2 inhibitors and cancer therapy: the long and winding road. Nat Rev Cancer. 2015; 15(10):577-92.
Chowdhury S, Xiu J, Ribeiro J R, Nicolaides T, Zhang J, Korn W M, et al. Consensus molecular subtyping of metastatic colorectal cancer expands biomarker-directed therapeutic benefit for patients with CMS1 and CMS2 tumors. Br J Cancer. 2024; 131(8):1328-39.
Cristescu R, Nebozhyn M, Zhang C, Albright A, Kobie J, Huang L, et al. Transcriptomic Determinants of Response to Pembrolizumab Monotherapy across Solid Tumor Types. Clin Cancer Res. 2022; 28(8):1680-9.
Dongre A, Weinberg R A. New insights into the mechanisms of epithelial-mesenchymal transition and implications for cancer. Nat Rev Mol Cell Biol. 2019; 20(2):69-84.
Dry J R, Pavey S, Pratilas C A, Harbron C, Runswick S, Hodgson D, et al. Transcriptional pathway signatures predict MEK addiction and response to selumetinib (AZD6244). Cancer Res. 2010; 70(6):2264-73.
Eide P W, Bruun J, Lothe R A, Sveen A. CMScaller: an R package for consensus molecular subtyping of colorectal cancer pre-clinical models. Sci Rep. 2017; 7(1):16618.
Farina-Sarasqueta A, van Lijnschoten G, Moerland E, Creemers G J, Lemmens V E, Rutten H J, et al. The BRAF V600E mutation is an independent prognostic factor for survival in stage II and stage III colon cancer patients. Annals of oncology: official journal of the European Society for Medical Oncology/ESMO. 2010; 21(12):2396-402.
Fenstermacher D A, Wenham R M, Rollison D E, Dalton W S. Implementing personalized medicine in a cancer center. Cancer journal. 2011; 17(6):528-36.
Frame M C. Src in cancer: deregulation and consequences for cell behaviour. Biochim Biophys Acta. 2002; 1602(2):114-30.
Galindo-Pumarino C, Collado M, Herrera M, Pena C. Tumor Microenvironment in Metastatic Colorectal Cancer: The Arbitrator in Patients' Outcome. Cancers (Basel). 2021; 13(5).
Gao Q, Liu W, Cai J, Li M, Gao Y, Lin W, et al. EphB2 promotes cervical cancer progression by inducing epithelial-mesenchymal transition. Human pathology. 2014; 45(2):372-81.
Guinney J, Dienstmann R, Wang X, de Reynies A, Schlicker A, Soneson C, et al. The consensus molecular subtypes of colorectal cancer. Nature medicine. 2015; 21(11):1350-6.
Herrera M, Islam A B, Herrera A, Martin P, Garcia V, Silva J, et al. Functional heterogeneity of cancer-associated fibroblasts from human colon tumors shows specific prognostic gene expression signature. Clin Cancer Res. 2013; 19(21):5914-26.
Holm S. A simple sequentially rejective multiple test procedure. Scandinavian Journal of Statistics. 1979; 6(2):65-70.
Joanito I, Wirapati P, Zhao N, Nawaz Z, Yeo G, Lee F, et al. Single-cell and bulk transcriptome sequencing identifies two epithelial tumor cell states and refines the consensus molecular classification of colorectal cancer. Nat Genet. 2022; 54(7):963-75.
Karhemo P R, Ravela S, Laakso M, Ritamo I, Tatti O, Makinen S, et al. An optimized isolation of biotinylated cell surface proteins reveals novel players in cancer metastasis. Journal of proteomics. 2012; 77:87-100.
Khambata-Ford S, Garrett C R, Meropol N J, Basik M, Harbison C T, Wu S, et al. Expression of epiregulin and amphiregulin and K-ras mutation status predict disease control in metastatic colorectal cancer patients treated with cetuximab. J Clin Oncol. 2007; 25(22):3230-7.
Lenz H J, Ou F S, Venook A P, Hochster H S, Niedzwiecki D, Goldberg R M, et al. Impact of Consensus Molecular Subtype on Survival in Patients With Metastatic Colorectal Cancer: Results From CALGB/SWOG 80405 (Alliance). J Clin Oncol. 2019; 37(22):1876-85.
Loboda A, Nebozhyn M V, Watters J W, Buser C A, Shaw P M, Huang P S, et al. EMT is the dominant program in human colon cancer. BMC Med Genomics. 2011; 4:9.
Malla S B, Byrne R M, Lafarge M W, Corry S M, Fisher N C, Tsantoulis P K, et al. Pathway level subtyping identifies a slow-cycling biological phenotype associated with poor clinical outcomes in colorectal cancer. Nat Genet. 2024; 56(3):458-72.
Marisa L, de Reynies A, Duval A, Selves J, Gaub M P, Vescovo L, et al. Gene expression classification of colon cancer into molecular subtypes: characterization, validation, and prognostic value. PLoS medicine. 2013; 10(5):e1001453.
Mesker W E, Junggeburt J M, Szuhai K, de Heer P, Morreau H, Tanke H J, et al. The carcinoma-stromal ratio of colon carcinoma is an independent factor for survival compared to lymph node status and tumor stage. Cell Oncol. 2007; 29(5):387-98.
Newman A M, Liu C L, Green M R, Gentles A J, Feng W, Xu Y, et al. Robust enumeration of cell subsets from tissue expression profiles. Nat Methods. 2015; 12(5):453-7.
Okita A, Takahashi S, Ouchi K, Inoue M, Watanabe M, Endo M, et al. Consensus molecular subtypes classification of colorectal cancer as a predictive factor for chemotherapeutic efficacy against metastatic colorectal cancer. Oncotarget. 2018; 9(27):18698-711.
Paauwe M, Schoonderwoerd M J A, Helderman R, Harryvan T J, Groenewoud A, van Pelt G W, et al. Endoglin Expression on Cancer-Associated Fibroblasts Regulates Invasion and Stimulates Colorectal Cancer Metastasis. Clin Cancer Res. 2018; 24(24):6331-44.
Pelka K, Hofree M, Chen J H, Sarkizova S, Pirl J D, Jorgji V, et al. Spatially organized multicellular immune hubs in human colorectal cancer. Cell. 2021; 184(18):4734-52 e20.
Picard E, Verschoor C P, Ma G W, Pawelec G. Relationships Between Immune Landscapes, Genetic Subtypes and Responses to Immunotherapy in Colorectal Cancer. Front Immunol. 2020; 11:369.
Quante M, Varga J, Wang T C, Greten F R. The gastrointestinal tumor microenvironment. Gastroenterology. 2013; 145(1):63-78.
Roelands J, Kuppen P J K, Vermeulen L, Maccalli C, Decock J, Wang E, et al. Immunogenomic Classification of Colorectal Cancer and Therapeutic Implications. Int J Mol Sci. 2017; 18(10).
Roth A D, Tejpar S, Delorenzi M, Yan P, Fiocca R, Klingbiel D, et al. Prognostic role of KRAS and BRAF in stage II and III resected colon cancer: results of the translational study on the PETACC-3, EORTC 40993, SAKK 60-00 trial. J Clin Oncol. 2010; 28(3):466-74.
Schell M J, Yang M, Missiaglia E, Delorenzi M, Soneson C, Yue B, et al. A Composite Gene Expression Signature Optimizes Prediction of Colorectal Cancer Metastasis and Outcome. Clin Cancer Res. 2016; 22(3):734-45.
Schell M J, Yang M, Teer J K, Lo F Y, Madan A, Coppola D, et al. A multigene mutation classification of 468 colorectal cancers reveals a prognostic role for APC. Nat Commun. 2016; 7:11743.
Schubbert S, Shannon K, Bollag G. Hyperactive Ras in developmental disorders and cancer. Nat Rev Cancer. 2007; 7(4):295-308.
Sclafani F, Kim T Y, Cunningham D, Kim T W, Tabernero J, Schmoll H J, et al. A Randomized Phase II/III Study of Dalotuzumab in Combination With Cetuximab and Irinotecan in Chemorefractory, KRAS Wild-Type, Metastatic Colorectal Cancer. J Natl Cancer Inst. 2015; 107(12):djv258.
Yaeger R, Yao Z, Hyman D M, Hechtman J F, Vakiani E, Zhao H, et al. Mechanisms of Acquired Resistance to BRAF V600E Inhibition in Colon Cancers Converge on RAF Dimerization and Are Sensitive to Its Inhibition. Cancer Res. 2017; 77(23):6513-23.
Yang M, Davis T B, Pflieger L, Nebozhyn M V, Loboda A, Wang H, et al. An integrative gene expression signature analysis identifies CMS4 KRAS-mutated colorectal cancers sensitive to combined MEK and SRC targeted therapy. BMC Cancer. 2022; 22(1):256.
Yang M, Schell M J, Loboda A, Nebozhyn M, Li J, Teer J K, et al. Repurposing EGFR Inhibitor Utility in Colorectal Cancer in Mutant APC and TP53 Subpopulations. Cancer Epidemiol Biomarkers Prev. 2019; 28(7):1141-52.
Yeatman T J. A renaissance for SRC. Nat Rev Cancer. 2004; 4(6):470-80.
Yu G, Gao Y, Ni C, Chen Y, Pan J, Wang X, et al. Reduced expression of EphB2 is significantly associated with nodal metastasis in Chinese patients with gastric cancer. Journal of cancer research and clinical oncology. 2011; 137(1):73-80.

What is claimed is:

1. A method of determining colorectal cancer (CRC) related therapy response, comprising:
obtaining a biological sample from a CRC subject;
measuring expression levels of a 10-gene tumor microenvironment signature (TME^{S}), a 10-gene epithelial signature (EPI^{S}), or a combination thereof in the biological sample;
calculating a TME^{s} score, EPI^{S} score or combination thereof;
classifying the CRC subject based on the TME^{s} score or the EPI^{S} score, wherein a high TME^{S} score indicates worse survival and resistance to CRC targeted therapy immunotherapy and MEK inhibitors, wherein a high EPI^{S} score indicates better survival and sensitivity to CRC targeted therapies; and
administering a therapeutically effective dose of CRC targeted therapy to the CRC subject.

2. The method of claim 1, wherein CRC targeted therapy comprises immunotherapy, EGFR inhibitors, SRC inhibitors or MEK inhibitors.

3. The method of claim 1, wherein the 10-gene epithelial signature is expressed in epithelial tumor cells or epithelial normal mucosa.

4. The method of claim 1, wherein the EPI^{S} score is calculated using gene expression levels of 10-gene epithelial signature.

5. The method of claim 1, wherein the 10-gene tumor microenvironment signature is expressed in stromal or immune cells in tumor microenvironment.

6. The method of claim 1, wherein the TME^{S} score is calculated using gene expression levels of 10-gene tumor microenvironment signature.

7. The method of claim 1, wherein the biological sample comprises a surgical resection specimen, tissue biopsy or fine needle aspirate.

8. The method of claim 1, wherein the 10-gene tumor microenvironment signature comprises CD109, AHNAK2, GAS1, PRKCDBP (CAVIN-3), MEIS2, NXN, GFPT2, PMP22, WWTR1 or PTRF (CAVIN-1) gene.

9. The method of claim 1, wherein the 10-gene epithelial signature comprises CDX1, CDX2, C10orf99, DDC, GPA33, FAM84A (LRATD1), NR1I2, MYB, C2orf89 (TRABD2A) or EPHB2 gene.

10. The method of claim 1, wherein the CRC subject is classified into at least one of consensus molecular subtypes (CMS) consisting of a CMS1 subtype, a CMS2 subtype, a CMS3 subtype, and a CMS4 subtype.

11. The method of claim 10, wherein CMS1 or CMS4 subtype is correlated to worse survival in the CRC subject.

12. The method of claim 10, wherein CMS2 or CMS3 subtype is correlated to better survival in the CRC subject.

13. The method of claim 10, wherein the CMS1 subtype and the CMS4 subtype is correlated with the TME^{s} score, wherein the CMS1 subtype is positively correlated with memory B cells, CD8+ T cells, gamma delta T cells, NK cells, macrophages or dendritic cells; and wherein the CMS4 subtype is positively correlated with stromal cells or tumor cells.

14. The method of claim 10, wherein the CMS2 subtype and the CMS3 subtype are correlated with the EPI^{s} score; wherein the CMS2 subtype and the CMS3 subtype are correlated with inactive B cells, resting NK cells and macrophages (M0).

15. A method for treating CRC in a subject, comprising:
obtaining a biological sample from the subject;
measuring expression levels of a 10-gene tumor microenvironment signature (TME^{S}), wherein the TME^{S} comprises CD109, AHNAK2, GAS1, PRKCDBP (CAVIN-3), MEIS2, NXN, GFPT2, PMP22, WWTR1 or PTRF (CAVIN-1) gene;
calculating a TME^{s} score;
analyzing presence of CRC subtype CMS4 or subtype CMS1 based on a high TME^{S} score; and
administering a therapeutically effective amount of immunotherapy, inhibitor therapy or cancer-associated fibroblast (CAF)-related therapy to the subject.

16. The method of claim 15, wherein the cancer-associated fibroblast (CAF)-related therapy comprises fibroblast activation protein (FAP) inhibitors, TGFβ inhibitors, or CXCL12/CXCR4 inhibitors.

17. A method for treating CRC in a subject, comprising:
obtaining a biological sample from the subject;
measuring expression levels of 10-gene epithelial-associated signature (EPI^{S}), wherein the EPI^{S} comprises CDX1, CDX2, C10orf99, DDC, GPA33, FAM84A (LRATD1), NR1I2, MYB, C2orf89 (TRABD2A) or EPHB2 gene;
calculating an EPI^{S} score;
analyzing presence of CRC subtype CMS2 or subtype CMS3 based on a high EPI^{S} score; and
administering a therapeutically effective amount of EGFR inhibitor (EGFRi) therapy to the subject.

18. The method of claim 17, wherein EGFR inhibitor therapy comprises cetuximab or panitumumab.
